(19) United States
(12) Patent Application Publication
(10) Pub. No.: US 2022/0281943 A1
(43) Pub. Date: Sep. 8, 2022

(54) CAR-T CELLS SPECIFIC FOR MODIFIED PROTEINS IN EXTRACELLULAR SPACES

(71) Applicant: The Regents of the University of California, Oakland, CA (US)

(72) Inventors: Jeffrey A. Bluestone, Oakland, CA (US); Caroline Raffin, Oakland, CA (US)

(21) Appl. No.: 17/635,054

(22) PCT Filed: Aug. 10, 2020
(86) PCT No.: PCT/US20/45603

§ 371 Date: Feb. 14, 2022

Related U.S. Application Data
(60) Provisional application No. 62/886,736, filed on Aug. 14, 2019.

(51) Int. Cl. C07K 14/725; C12N 5/0783; C07K 16/18

(52) U.S. Cl. CPC C07K 14/7051; C12N 5/0637; C07K 16/18; C12N 2510/00; C07K 2317/24; C07K 2317/622; C07K 2319/03

(57) ABSTRACT

Chimeric antigen receptor (CAR)-expressing Tregs specifically target modified protein or proteins present in the extra cellular matrix of an inflammatory lesion of patients to induce a localized and effective immunosuppressive response.

### CROSS-REFERENCE TO RELATED APPLICATIONS

This application claims the benefit of priority under 35 U.S.C. § 119(e) to U.S. Provisional Application No. 62/886,736, filed on Aug. 14, 2019, which is incorporated herein by reference in its entirety.

### FIELD

The present disclosure relates to chimeric antigen receptors (CAR), chimeric antigen receptor T cells (CAR-T) and use in treatment of diseases, such as autoimmunity, brain dementias and other inflammatory diseases.

### BACKGROUND OF THE DISCLOSURE

Traditionally, antigen-specific T-cells (Treg) have been generated by selective expansion of peripheral blood T-cells natively specific for the target antigen. It has been shown that regulatory T cells can have a profound effect on a variety of inflammatory diseases ranging from autoimmunity to transplantation to inflammation-based diseases such as frontotemporal dementias, heart disease, diabetes, etc. Moreover, Tregs have two distinct properties that enhance their activity. First, through a variety of mechanisms, they exhibit bystander suppression, namely the ability to suppress locally once activated even if the target antigen is not expressed directly on the affected cells in a tissue. Second, Tregs exhibit infectious tolerance, namely, the ability, through their suppressive activities to “educate” other cells in their local environment to become suppressor cells which amplify the effectiveness of the Treg and the efficacy can be achieved using monoclonal or pauci-clonal Treg populations, with regards to antigen specificity. However, it has proven difficult and quite often impossible to select and expand large numbers of T-cells specific for most cancer and autoantigens.

This has led to the development of therapeutic opportunities where in Tregs are isolated from individuals, modified with specific T cell receptor (TCR) and Chimeric Antigen Receptors (CAR) specific for tissue-specific and pathogenic antigens, expanded and reinfused into patients to ameliorate disease. Preclinical studies have shown the efficacy of this approach and current efforts are underway to exploit current gene editing technologies to develop cell therapies in humans. However, there remains some key concerns with the approach, including the fact that among the activities of Tregs is the potential for these cells to lyse target cells expressing the target antigen (a function evolved by the cells to help eliminate antigen presenting cells). Thus, in some setting, it remains possible that rather than suppressing immunity, Tregs could destroy the tissue cells that are supported to be protected.

### SUMMARY

The disclosure is directed, in part, to chimeric antigen receptors (CAR) which specifically recognize antigens associated with autoimmune diseases. In particular, the CAR are specific for post-translationally modified antigens. The CARs are transduced into T cells, such as, regulatory T cells, which suppress the autoimmune response or cytotoxic T cells.

Accordingly, in one aspect of the present disclosure there is provided a chimeric antigen receptor (CAR) including an antigen specific binding domain, a hinge domain, a transmembrane domain, co-stimulatory domain, and a primary signaling domain, optionally derived from a CD3 chain domain, wherein the antigen specific binding domain specifically binds to a modified protein, peptide or fragments thereof.

In a second aspect of the present disclosure there is provided an isolated T cell that is modified to express: a chimeric antigen receptor (CAR) including an antigen binding domain linked to at least one co-stimulatory domain and a primary signaling domain, optionally derived from a CD3 chain domain, wherein the antigen binding domain specifically binds to an modified protein present in the extracellular space of the inflammatory lesion.

In a third aspect of the present disclosure there is provided a method of treating a subject diagnosed with an inflammatory disease, including (i) identifying a modified protein present in the extracellular space of the inflammatory lesion; (ii) generating a chimeric antigen receptor (CAR) that binds to the modified protein of step (i); (iii) expressing the CAR of step (ii) in an isolated T cell; (iv) administering the isolated T of step (iii) to a subject.

In a fourth aspect of the present disclosure there is provided an expression vector encoding the CAR provided herein including embodiments thereof.

In a fifth aspect of the present disclosure there is provided an isolated cell comprising an expression vector provided herein including embodiments thereof.

In a sixth aspect of the present disclosure there is provided a pharmaceutical composition comprising a CAR, an isolated T cell, an expression vector, or a host cell provided herein including embodiments thereof.

Other aspects are described infra.

### Definitions

Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

As used herein, the terms “comprising,” “comprise” or “comprised,” and variations thereof, in reference to defined or described elements of an item, composition, apparatus, method, process, system, etc. are meant to be inclusive or open ended, permitting additional elements, thereby indicating that the defined or described item, composition, apparatus, method, process, system, etc. includes those specified elements—or, as appropriate, equivalents thereof—and that other elements can be included and still fall within the scope/definition of the defined item, composition, apparatus, method, process, system, etc.

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

As used in this specification and the appended claims, the term “or” is generally employed in its sense including “and/or” unless the content clearly dictates otherwise.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value or range. Alternatively, particularly with respect to biological systems or processes, the term can mean within an order of magnitude within 5-fold, and also within 2-fold, of a value. Where particular values are described in the application and claims, unless otherwise stated the term “about” meaning within an acceptable error range for the particular value should be assumed.

As used herein, the term “affinity” is meant as a measure of binding strength. Without being bound to theory, affinity depends on the closeness of stereochemical fit between antibody combining sites and antigen determinants, on the size of the area of contact between them, and on the distribution of charged and hydrophobic groups. Affinity also includes the term “avidity,” which refers to the strength of the antigen-antibody bond after formation of reversible complexes. Methods for calculating the affinity of an antibody for an antigen are known in the art, including use of binding experiments to calculate affinity. Antibody activity in functional assays (e.g., flow cytometry assay) is also reflective of antibody affinity. Antibodies and affinities can be phenotypically characterized and compared using functional assays (e.g., flow cytometry assay).

As used herein, the term “agent” is meant to encompass any molecule, chemical entity, composition, drug, therapeutic agent, chemotherapeutic agent, or biological agent capable of preventing, ameliorating, or treating a disease or other medical condition. The term includes small molecule compounds, antisense oligonucleotides, siRNA reagents, antibodies, antibody fragments bearing epitope recognition sites, such as Fab, Fab′, F(ab′)₂ fragments, Fv fragments, single chain antibodies, antibody mimetics (such as DARPins, affibody molecules, affilins, affitins, anticalins, avimers, fynomers, Kunitz domain peptides and monobodies), peptoids, aptamers; enzymes, peptides organic or inorganic molecules, natural or synthetic compounds and the like. An agent can be assayed in accordance with the methods of the disclosure at any stage during clinical trials, during pre-trial testing, or following FDA-approval.

By “ameliorate” is meant decrease, suppress, attenuate, diminish, arrest, or stabilize the development or progression of a disease.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “polypeptide,” “peptide” and “protein” are used interchangeably herein to refer to a polymer of amino acid residues, wherein the polymer may, in certain embodiments, be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety.

As may be used herein, the terms “nucleic acid,” “nucleic acid molecule,” “nucleic acid oligomer,” “oligonucleotide,” “nucleic acid sequence,” “nucleic acid fragment” and “polynucleotide” are used interchangeably and are intended to include, but are not limited to, a polymeric form of nucleotides covalently linked together that may have various lengths, either deoxyribonucleotides or ribonucleotides, or analogs, derivatives or modifications thereof. Different polynucleotides may have different three-dimensional structures, and may perform various functions, known or unknown. Non-limiting examples of polynucleotides include a gene, a gene fragment, an exon, an intron, intergenic DNA (including, without limitation, heterochromatic DNA), messenger RNA (mRNA), transfer RNA, ribosomal RNA, a ribozyme, cDNA, a recombinant polynucleotide, a branched polynucleotide, a plasmid, a vector, isolated DNA of a sequence, isolated RNA of a sequence, a nucleic acid probe, and a primer. Polynucleotides useful in the methods of the disclosure may comprise natural nucleic acid sequences and variants thereof, artificial nucleic acid sequences, or a combination of such sequences.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the disclosure.

The following eight groups each contain amino acids that are conservative substitutions for one another:

### 1) Alanine (A), Glycine (G);

2) Aspartic acid (D), Glutamic acid (E);

### 3) Asparagine (N), Glutamine (Q);

### 4) Arginine (R), Lysine (K);

### 5) Isoleucine (I), Leucine (L), Methionine (M), Valine (V);

### 6) Phenylalanine (F), Tyrosine (Y), Tryptophan (W);

### 7) Serine (S), Threonine (T); and

### 8) Cysteine (C), Methionine (M)

(see, e.g., Creighton, Proteins (1984)).

The term “bispecific T-cell engager (BiTE)”, “BiTe” or “bispecific antibody” as provided herein is used according to its conventional meaning well known in the art and refers to a bispecific recombinant protein capable to simultaneously bind to two different antigens. In contrast to traditional monoclonal antibodies, BiTE antibodies consist of two independently different antibody regions (e.g., two single-chain variable fragments (scFv)), each of which binds a different antigen. One antibody region engages effector cells (e.g., T cells) by binding an effector cell-specific antigen (e.g., CD3 molecule) and the second antibody region binds a target antigen (e.g., extracellular inflammatory antigen). Binding of the BiTE to the two antigens will link the effector cell (e.g., T cell) to the target cell (e.g., tumor cell) and activate the effector cell (e.g., T cell) via effector cell-specific antigen signaling (e.g., CD3 signaling). The activated effector cell (e.g., T cell) will then exert cytotoxic activity against the target cell (e.g., tumor cells).

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence.

“Nucleic acid” refers to nucleotides (e.g., deoxyribonucleotides or ribonucleotides) and polymers thereof in either single-, double- or multiple-stranded form, or complements thereof; or nucleosides (e.g., deoxyribonucleosides or ribonucleosides). In embodiments, “nucleic acid” does not include nucleosides. The terms “polynucleotide,” “oligonucleotide,” “oligo” or the like refer, in the usual and customary sense, to a linear sequence of nucleotides. The term “nucleoside” refers, in the usual and customary sense, to a glycosylamine including a nucleobase and a five-carbon sugar (ribose or deoxyribose). Non limiting examples, of nucleosides include, cytidine, uridine, adenosine, guanosine, thymidine and inosine. The term “nucleotide” refers, in the usual and customary sense, to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA. Examples of nucleic acid, e.g. polynucleotides contemplated herein include any types of RNA, e.g. mRNA, siRNA, miRNA, and guide RNA and any types of DNA, genomic DNA, plasmid DNA, and minicircle DNA, and any fragments thereof. The term “duplex” in the context of polynucleotides refers, in the usual and customary sense, to double strandedness. Nucleic acids can be linear or branched. For example, nucleic acids can be a linear chain of nucleotides or the nucleic acids can be branched, e.g., such that the nucleic acids comprise one or more arms or branches of nucleotides. Optionally, the branched nucleic acids are repetitively branched to form higher ordered structures such as dendrimers and the like.

Nucleic acids, including e.g., nucleic acids with a phosphothioate backbone, can include one or more reactive moieties. As used herein, the term reactive moiety includes any group capable of reacting with another molecule, e.g., a nucleic acid or polypeptide through covalent, non-covalent or other interactions. By way of example, the nucleic acid can include an amino acid reactive moiety that reacts with an amino acid on a protein or polypeptide through a covalent, non-covalent or other interaction.

The terms also encompass nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphodiester derivatives including, e.g., phosphoramidate, phosphorodiamidate, phosphorothioate (also known as phosphothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see Eckstein, OLIGONUCLEOTIDES AND ANALOGUES: A PRACTICAL APPROACH, Oxford University Press) as well as modifications to the nucleotide bases such as in 5-methyl cytidine or pseudouridine; and peptide nucleic acid backbones and linkages. Other analog nucleic acids include those with positive backbones; non-ionic backbones, modified sugars, and non-ribose backbones (e.g. phosphorodiamidate morpholino oligos or locked nucleic acids (LNA) as known in the art), including those described in U.S. Pat. Nos. 5,235,033 and 5,034,506, and Chapters 6 and 7, ASC Symposium Series 580, CARBOHYDRATE MODIFICATIONS IN ANTISENSE RESEARCH, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In embodiments, the internucleotide linkages in DNA are phosphodiester, phosphodiester derivatives, or a combination of both.

Nucleic acids can include nonspecific sequences. As used herein, the term “nonspecific sequence” refers to a nucleic acid sequence that contains a series of residues that are not designed to be complementary to or are only partially complementary to any other nucleic acid sequence. By way of example, a nonspecific nucleic acid sequence is a sequence of nucleic acid residues that does not function as an inhibitory nucleic acid when contacted with a cell or organism.

As used herein, the term “antibody” means not only intact antibody molecules, but also fragments of antibody molecules that retain immunogen-binding ability. Such fragments are also well known in the art and are regularly employed both in vitro and in vivo. Accordingly, as used herein, the term “antibody” means not only intact immunoglobulin molecules but also the well-known active fragments F(ab′)₂, and Fab. F(ab′)₂, and Fab fragments that lack the Fc fragment of intact antibody, clear more rapidly from the circulation, and may have less non-specific tissue binding of an intact antibody (Wahl et al., J. Nucl. Med. 24:316-325 (1983). The antibodies of the disclosure comprise whole native antibodies, bispecific antibodies; chimeric antibodies; Fab, Fab′, single chain V region fragments (scFv), fusion polypeptides, and unconventional antibodies.

The phrase “specifically (or selectively) binds” to an antibody or “specifically (or selectively) immunoreactive with,” when referring to a protein or peptide, refers to a binding reaction that is determinative of the presence of the protein, often in a heterogeneous population of proteins and other biologics. Thus, under designated immunoassay conditions, the specified antibodies bind to a particular protein at least two times the background and more typically more than 10 to 100 times background. Specific binding to an antibody under such conditions requires an antibody that is selected for its specificity for a particular protein. For example, polyclonal antibodies can be selected to obtain only a subset of antibodies that are specifically immunoreactive with the selected antigen and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Using Antibodies, A Laboratory Manual (1998) for a description of immunoassay formats and conditions that can be used to determine specific immunoreactivity).

An exemplary immunoglobulin (antibody) structural unit comprises a tetramer. Each tetramer is composed of two identical pairs of polypeptide chains, each pair having one “light” (about 25 kDa) and one “heavy” chain (about 50-70 kDa). The N-terminus of each chain defines a variable region of about 100 to 110 or more amino acids primarily responsible for antigen recognition. The terms “variable heavy chain,” “VH,” or “VH” refer to the variable region of an immunoglobulin heavy chain, including an Fv, scFv, dsFv or Fab; while the terms “variable light chain,” “VL” or “VL” refer to the variable region of an immunoglobulin light chain, including of an Fv, scFv, dsFv or Fab.

Examples of antibody functional fragments include, but are not limited to, complete antibody molecules, antibody fragments, such as Fv, single chain Fv (scFv), complementarity determining regions (CDRs), VL (light chain variable region), VH (heavy chain variable region), Fab, F(ab)2′ and any combination of those or any other functional portion of an immunoglobulin peptide capable of binding to target antigen (see, e.g., FUNDAMENTAL IMMUNOLOGY (Paul ed., 4th ed. 2001). As appreciated by one of skill in the art, various antibody fragments can be obtained by a variety of methods, for example, digestion of an intact antibody with an enzyme, such as pepsin; or de novo synthesis. Antibody fragments are often synthesized de novo either chemically or by using recombinant DNA methodology. Thus, the term antibody, as used herein, includes antibody fragments either produced by the modification of whole antibodies, or those synthesized de novo using recombinant DNA methodologies (e.g., single chain Fv) or those identified using phage display libraries (see, e.g., McCafferty et al., (1990) Nature 348:552). The term “antibody” also includes bivalent or bispecific molecules, diabodies, triabodies, and tetrabodies. Bivalent and bispecific molecules are described in, e.g., Kostelny et al. (1992) J. Immunol. 148:1547, Pack and Pluckthun (1992) Biochemistry 31:1579, Hollinger et al. (1993), PNAS. USA 90:6444, Gruber et al. (1994) J Immunol. 152:5368, Zhu et al. (1997) Protein Sci. 6:781, Hu et al. (1996) Cancer Res. 56:3055, Adams et al. (1993) Cancer Res. 53:4026, and McCartney, et al. (1995) Protein Eng. 8:301.

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity. The preferred antibodies of, and for use according to the disclosure include humanized and/or chimeric monoclonal antibodies.

The term “chimeric antigen receptor” or “CAR” as used herein refers to an antigen-binding domain that is fused to an intracellular signaling domain capable of activating or stimulating an immune cell, and in certain embodiments, the CAR also comprises a transmembrane domain. In certain embodiments the CAR's extracellular antigen-binding domain is composed of a single chain variable fragment (scFv) derived from fusing the variable heavy and light regions of a murine or humanized monoclonal antibody. Alternatively, scFvs may be used that are derived from Fab's (instead of from an antibody, e.g., obtained from Fab libraries). In various embodiments, the scFv is fused to the transmembrane domain and then to the intracellular signaling domain. “First-generation” CARs include those that solely provide CD3ζ signals upon antigen binding, “Second-generation” CARs include those that provide both co-stimulation (e.g., CD28 or CD137) and activation (CD3ζ). “Third-generation” CARs include those that provide multiple co-stimulation (e.g. CD28 and CD137) and activation (CD3ζ). A fourth generation of CARs have been described, CAR T cells redirected for cytokine killing (TRUCKS) where the vector containing the CAR construct possesses a cytokine cassette. When the CAR is ligated, the CAR T cell deposits a pro-inflammatory cytokine into the tumor lesion. A CAR-T cell is a T cell that expresses a chimeric antigen receptor. The phrase “chimeric antigen receptor (CAR),” as used herein and generally used in the art, refers to a recombinant fusion protein that has an antigen-specific extracellular domain coupled to an intracellular domain that directs the cell to perform a specialized function upon binding of an antigen to the extracellular domain. The terms “artificial T-cell receptor,” “chimeric T-cell receptor,” and “chimeric immunoreceptor” may each be used interchangeably herein with the term “chimeric antigen receptor.”

The term “associated” or “associated with” in the context of a substance or substance activity or function associated with a disease (e.g., inflammatory disease, autoimmune disease) means that the disease (e.g. inflammatory disease, autoimmune disease) is caused by (in whole or in part), or a symptom of the disease is caused by (in whole or in part) the substance or substance activity or function. As used herein, what is described as being associated with a disease, if a causative agent, could be a target for treatment of the disease.

“Diagnostic” or “diagnosed” means identifying the presence or nature of a pathologic condition. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay, are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, it suffices if the method provides a positive indication that aids in diagnosis.

A “disease” is a state of health of an animal wherein the animal cannot maintain homeostasis, and wherein if the disease is not ameliorated then the animal's health continues to deteriorate. Examples of diseases include autoimmune diseases such as, rheumatoid arthritis (RA), inflammatory bowel disease (IBD), Crohn's disease (CD), ankylo sing spondylitis (AS), Alzheimer disease, Fronotemporal Dementia, Duchenne's muscular dystrophy, Parkinson's disease.

As used herein, the term “inflammatory disease” refers to a disease or condition characterized by aberrant inflammation (e.g. an increased level of inflammation compared to a control such as a healthy person not suffering from a disease). Examples of inflammatory diseases include autoimmune diseases, arthritis, rheumatoid arthritis, psoriatic arthritis, juvenile idiopathic arthritis, multiple sclerosis, systemic lupus erythematosus (SLE), myasthenia gravis, juvenile onset diabetes, diabetes mellitus type 1, Guillain-Barre syndrome, Hashimoto's encephalitis, Hashimoto's thyroiditis, ankylosing spondylitis, psoriasis, Sjogren's syndrome, vasculitis, glomerulonephritis, auto-immune thyroiditis, Behcet's disease, Crohn's disease, ulcerative colitis, bullous pemphigoid, sarcoidosis, ichthyosis, Graves ophthalmopathy, inflammatory bowel disease, Addison's disease, Vitiligo, asthma, allergic asthma, acne vulgaris, celiac disease, chronic prostatitis, inflammatory bowel disease, pelvic inflammatory disease, reperfusion injury, ischemia reperfusion injury, stroke, sarcoidosis, transplant rejection, interstitial cystitis, atherosclerosis, scleroderma, and atopic dermatitis.

As used herein, the term “autoimmune disease” refers to a disease or condition in which a subject's immune system has an aberrant immune response against a substance that does not normally elicit an immune response in a healthy subject. Examples of autoimmune diseases that may be treated with a compound, pharmaceutical composition, or method described herein include Acute Disseminated Encephalomyelitis (ADEM), Acute necrotizing hemorrhagic leukoencephalitis, Addison's disease, Agammaglobulinemia, Alopecia areata, Amyloidosis, Ankylosing spondylitis, Anti-GBM/Anti-TBM nephritis, Antiphospholipid syndrome (APS), Autoimmune angioedema, Autoimmune aplastic anemia, Autoimmune dysautonomia, Autoimmune hepatitis, Autoimmune hyperlipidemia, Autoimmune immunodeficiency, Autoimmune inner ear disease (AIED), Autoimmune myocarditis, Autoimmune oophoritis, Autoimmune pancreatitis, Autoimmune retinopathy, Autoimmune thrombocytopenic purpura (ATP), Autoimmune thyroid disease, Autoimmune urticaria, Axonal or neuronal neuropathies, Balo disease, Behcet's disease, Bullous pemphigoid, Cardiomyopathy, Castleman disease, Celiac disease, Chagas disease, Chronic fatigue syndrome, Chronic inflammatory demyelinating polyneuropathy (CIDP), Chronic recurrent multifocal ostomyelitis (CRMO), Churg-Strauss syndrome, Cicatricial pemphigoid/benign mucosal pemphigoid, Crohn's disease, Cogans syndrome, Cold agglutinin disease, Congenital heart block, Coxsackie myocarditis, CREST disease, Essential mixed cryoglobulinemia, Demyelinating neuropathies, Dermatitis herpetiformis, Dermatomyositis, Devic's disease (neuromyelitis optica), Discoid lupus, Dressler's syndrome, Endometriosis, Eosinophilic esophagitis, Eosinophilic fasciitis, Erythema nodosum, Experimental allergic encephalomyelitis, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis (GPA) (formerly called Wegener's Granulomatosis), Graves' disease, Guillain-Barre syndrome, Hashimoto's encephalitis, Hashimoto's thyroiditis, Hemolytic anemia, Henoch-Schonlein purpura, Herpes gestationis, Hypogammaglobulinemia, Idiopathic thrombocytopenic purpura (ITP), IgA nephropathy, IgG4-related sclerosing disease, Immunoregulatory lipoproteins, Inclusion body myositis, Interstitial cystitis, Juvenile arthritis, Juvenile diabetes (Type 1 diabetes), Juvenile myositis, Kawasaki syndrome, Lambert-Eaton syndrome, Leukocytoclastic vasculitis, Lichen planus, Lichen sclerosus, Ligneous conjunctivitis, Linear IgA disease (LAD), Lupus (SLE), Lyme disease, chronic, Meniere's disease, Microscopic polyangiitis, Mixed connective tissue disease (MCTD), Mooren's ulcer, Mucha-Habermann disease, Multiple sclerosis, Myasthenia gravis, Myositis, Narcolepsy, Neuromyelitis optica (Devic's), Neutropenia, Ocular cicatricial pemphigoid, Optic neuritis, Palindromic rheumatism, PANDAS (Pediatric Autoimmune Neuropsychiatric Disorders Associated with Streptococcus), Paraneoplastic cerebellar degeneration, Paroxysmal nocturnal hemoglobinuria (PNH), Parry Romberg syndrome, Parsonnage-Turner syndrome, Pars planitis (peripheral uveitis), Pemphigus, Peripheral neuropathy, Perivenous encephalomyelitis, Pernicious anemia, POEMS syndrome, Polyarteritis nodosa, Type I, II, & III autoimmune polyglandular syndromes, Polymyalgia rheumatica, Polymyositis, Postmyocardial infarction syndrome, Postpericardiotomy syndrome, Progesterone dermatitis, Primary biliary cirrhosis, Primary sclerosing cholangitis, Psoriasis, Psoriatic arthritis, Idiopathic pulmonary fibrosis, Pyoderma gangrenosum, Pure red cell aplasia, Raynauds phenomenon, Reactive Arthritis, Reflex sympathetic dystrophy, Reiter's syndrome, Relapsing polychondritis, Restless legs syndrome, Retroperitoneal fibrosis, Rheumatic fever, Rheumatoid arthritis, Sarcoidosis, Schmidt syndrome, Scleritis, Scleroderma, Sjogren's syndrome, Sperm & testicular autoimmunity, Stiff person syndrome, Subacute bacterial endocarditis (SBE), Susac's syndrome, Sympathetic ophthalmia, Takayasu's arteritis, Temporal arteritis/Giant cell arteritis, Thrombocytopenic purpura (TTP), Tolosa-Hunt syndrome, Transverse myelitis, Type 1 diabetes, Ulcerative colitis, Undifferentiated connective tissue disease (UCTD), Uveitis, Vasculitis, Vesiculobullous dermatosis, Vitiligo, or Wegener's granulomatosis (i.e., Granulomatosis with Polyangiitis (GPA).

As used herein, the term “neurodegenerative disorder” refers to a disease or condition in which the function of a subject's nervous system becomes impaired. Examples of neurodegenerative diseases that may be treated with a compound, pharmaceutical composition, or method described herein include Alexander's disease, Alper's disease, Alzheimer's disease, Amyotrophic lateral sclerosis, Ataxia telangiectasia, Batten disease (also known as Spielmeyer-Vogt-Sjogren-Batten disease), Bovine spongiform encephalopathy (BSE), Canavan disease, chronic fatigue syndrome, Cockayne syndrome, Corticobasal degeneration, Creutzfeldt-Jakob disease, frontotemporal dementia, Gerstmann-Sträussler-Scheinker syndrome, Huntington's disease, HIV-associated dementia, Kennedy's disease, Krabbe's disease, kuru, Lewy body dementia, Machado-Joseph disease (Spinocerebellar ataxia type 3), Multiple sclerosis, Multiple System Atrophy, myalgic encephalomyelitis, Narcolepsy, Neuroborreliosis, Parkinson's disease, Pelizaeus-Merzbacher Disease, Pick's disease, Primary lateral sclerosis, Prion diseases, Refsum's disease, Sandhoff s disease, Schilder's disease, Subacute combined degeneration of spinal cord secondary to Pernicious Anaemia, Schizophrenia, Spinocerebellar ataxia (multiple types with varying characteristics), Spinal muscular atrophy, Steele-Richardson-Olszewski disease, progressive supranuclear palsy, or Tabes dorsalis.

The terms “domain” and “motif”, used interchangeably herein, refer to both structured domains having one or more particular functions and unstructured segments of a polypeptide that, although unstructured, retain one or more particular functions. For example, a structured domain may encompass but is not limited to a continuous or discontinuous plurality of amino acids, or portions thereof, in a folded polypeptide that comprise a three-dimensional structure which contributes to a particular function of the polypeptide. In other instances, a domain may include an unstructured segment of a polypeptide comprising a plurality of two or more amino acids, or portions thereof, that maintains a particular function of the polypeptide unfolded or disordered. Also encompassed within this definition are domains that may be disordered or unstructured but become structured or ordered upon association with a target or binding partner. Non-limiting examples of intrinsically unstructured domains and domains of intrinsically unstructured proteins are described, e.g., in Dyson & Wright. Nature Reviews Molecular Cell Biology 6:197-208 (2005).

The term “hinge” or “hinge region” refers to a flexible connector region, e.g. natural or synthetic polypeptides, or any other type of molecule, providing structural flexibility and spacing to flanking polypeptide regions.

The term “linker”, also referred to as a “spacer” or “spacer domain” as used herein, refers to a an amino acid or sequence of amino acids that that is optionally located between two amino acid sequences in a fusion protein of the disclosure.

“Parenteral” administration of an immunogenic composition includes, e.g., subcutaneous (s.c.), intravenous (i.v.), intramuscular (i.m.), or intrasternal injection, or infusion techniques.

The terms “patient” or “individual” or “subject” are used interchangeably herein, and refers to a mammalian subject to be treated, with human patients being preferred. In some cases, the methods of the disclosure find use in experimental animals, in veterinary application, and in the development of animal models for disease, including, but not limited to, rodents including mice, rats, and hamsters, and primates.

As used herein, the term “single-chain variable fragment” or “scFv” is a fusion protein of the variable regions of the heavy (VH) and light chains (VL) of an immunoglobulin covalently linked to form a VH::VL heterodimer. The heavy (VH) and light chains (VL) are either joined directly or joined by a peptide-encoding linker (e.g., 10, 15, 20, 25 amino acids), which connects the N-terminus of the VH with the C-terminus of the VL, or the C-terminus of the VH with the N-terminus of the VL. The linker is usually rich in glycine for flexibility, as well as serine or threonine for solubility. Despite removal of the constant regions and the introduction of a linker, scFv proteins retain the specificity of the original immunoglobulin. Single chain Fv polypeptide antibodies can be expressed from a nucleic acid including VH- and VL-encoding sequences as described by Huston, et al. (Proc. Nat. Acad. Sci. USA, 85:5879-5883, 1988). See, also, U.S. Pat. Nos. 5,091,513, 5,132,405 and 4,956,778; and U.S. Patent Publication Nos. 20050196754 and 20050196754. Antagonistic scFvs having inhibitory activity have been described (see, e.g., Zhao et al., Hyrbidoma (Larchmt) 2008 27(6):455-51; Peter et al., J Cachexia Sarcopenia Muscle 2012 Aug. 12; Shieh et al., J Imunol 2009 183(4):2277-85; Giomarelli et al., Thromb Haemost 2007 97(6):955-63; Fife et al., J Clin Invst 2006 116(8):2252-61; Brocks et al., Immunotechnology 1997 3(3): 173-84; Moosmayer et al., Ther Immunol 1995 2(10:31-40). Agonistic scFvs having stimulatory activity have been described (see, e.g., Peter et al., J Bioi Chem 2003 25278(38):36740-7; Xie et al., Nat Biotech 1997 15(8):768-71; Ledbetter et al., Crit Rev Immunol 1997 17(5-6):427-55; Ho et al., BioChim Biophys Acta 2003 1638(3):257-66).

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

A “vector” is a composition of matter which comprises an isolated nucleic acid and which can be used to deliver the isolated nucleic acid to the interior of a cell. Examples of vectors include but are not limited to, linear polynucleotides, polynucleotides associated with ionic or amphiphilic compounds, plasmids, and viruses. Thus, the term “vector” includes an autonomously replicating plasmid or a virus. The term is also construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

All genes, gene names, and gene products disclosed herein are intended to correspond to homologs from any species for which the compositions and methods disclosed herein are applicable. Thus, the terms include, but are not limited to genes and gene products from humans and mice. It is understood that when a gene or gene product from a particular species is disclosed, this disclosure is intended to be exemplary only, and is not to be interpreted as a limitation unless the context in which it appears clearly indicates. Thus, for example, for the genes or gene products disclosed herein, which in some embodiments relate to mammalian nucleic acid and amino acid sequences, are intended to encompass homologous and/or orthologous genes and gene products from other animals including, but not limited to other mammals, fish, amphibians, reptiles, and birds. In preferred embodiments, the genes, nucleic acid sequences, amino acid sequences, peptides, polypeptides and proteins are human. The term “gene” is also intended to include variants.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

The practice of the present disclosure employs, unless otherwise indicated, conventional techniques of chemistry, molecular biology, microbiology, recombinant DNA, genetics, immunology, cell biology, cell culture and transgenic biology, which are within the skill of the art. See, e.g., Maniatis et al., 1982, Molecular Cloning (Cold Spring Harbor Laboratory Press, Cold Spring Harbor, N.Y.); Sambrook et al., 1989, Molecular Cloning, 2nd Ed. (Cold Spring Harbor Laboratory Press, Cold Spring Harbor, N.Y.); Sambrook and Russell, 2001, Molecular Cloning, 3rd Ed. (Cold Spring Harbor Laboratory Press, Cold Spring Harbor, N.Y.); Ausubel et al., 1992), Current Protocols in Molecular Biology (John Wiley & Sons, including periodic updates); Glover, 1985, DNA Cloning (IRL Press, Oxford); Anand, 1992; Guthrie and Fink, 1991; Harlow and Lane, 1988, Antibodies, (Cold Spring Harbor Laboratory Press, Cold Spring Harbor, N.Y.); Jakoby and Pastan, 1979; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Riott, Essential Immunology, 6th Edition, Blackwell Scientific Publications, Oxford, 1988; Hogan et al., Manipulating the Mouse Embryo, (Cold Spring Harbor Laboratory Press, Cold Spring Harbor, N.Y., 1986); Westerfield, M., The zebrafish book. A guide for the laboratory use of zebrafish (Danio rerio), (4th Ed., Univ. of Oregon Press, Eugene, 2000).

A “lentivirus” as used herein refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses.

The terms “multiplicity of infection” or “MOI” are used according to its plain ordinary meaning in Virology and refers to the ratio of components (e.g., poxvirus) to the target (e.g, cell) in a given area. In embodiments, the area is assumed to be homogenous.

The term “replicate” is used in accordance with its plain ordinary meaning and refers to the ability of a cell to produce progeny. A person of ordinary skill in the art will immediately understand that the term replicate when used in connection with DNA, refers to the biological process of producing two identical replicas of DNA from one original DNA molecule.

### BRIEF DESCRIPTION OF THE DRAWINGS

FIG. 1 is a schematic representation of CAR-Treg based adoptive cell transfer therapy manufacturing process.

### DETAILED DESCRIPTION

The present disclosure is directed to chimeric antigen receptors (CARs) targeting modified proteins specifically located in the extracellular interstitial space of unwarranted inflammation in patients with autoimmune diseases (e.g. Rheumatoid Arthritis, Multiple Sclerosis, Type 1 Diabetes, Vitiligo, Pemphigus, Systemic Lupus Erythematosus), organ transplants and non-autoimmune inflammatory disorders (e.g. Alzheimer's disease, Frontotemporal Dementias, Duchenne's Muscular Dystrophy, Parkinson's disease, Chronic Obstructive Pulmonary Disease, Cardiovascular disease). These extracellular/modified protein-specific CAR are then used to redirect the antigen specificity of immune regulatory cells, such as regulatory T cells (Tregs), to target the inflamed tissue microenvironment rather than the tissue cells. The addition of an antigen specificity to a protein specifically located in the extracellular space at the site of the disease aims to improve the potency of the engineered immune regulatory cells to suppress unwarranted immune response and reduce the risk of pan-immunosuppression while preventing cell-contact-mediated tissue destruction. Importantly, the presence of the target protein in the extracellular space may be the consequence of secretion, cell death, neutrophil extracellular trap formation or extracellular modification. Moreover, modified proteins correspond to proteins that underwent rather post-translational modification (e.g. citrullination, ubiquitination, amidation), or denaturation, aggregation, or any modification that leads to the generation of novel epitopes possibly recognized by a unique set of antibodies.

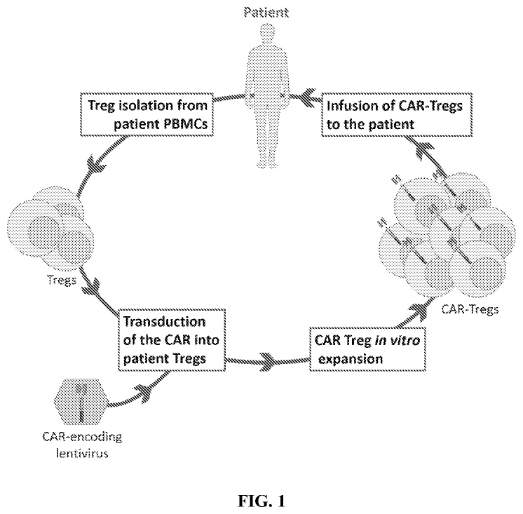

In certain embodiments, adoptive cell therapy (ACT) is used to reinfuse the CAR-T cells. For example, Tregs from the patient (autologous) are isolated from the peripheral blood. Then, the isolated Tregs will be genetically reengineered using the lentiviral vector encoding for a CAR transgene of interest. CAR Tregs will undergo two rounds of expansion in vitro and then be infused to the patient (see FIG. 1).

### T-Cell Receptors

The chains of the T-cell antigen receptor (TCR) of a T-cell clone are each composed of a unique combination of domains designated variable (V), [diversity (D),] joining (J), and constant (C). In each T-cell clone, the combination of V, D, and J domains of both the alpha and the beta chains or of both the delta and gamma chains participates in antigen recognition in a manner which is uniquely characteristic of that T-cell clone and defines a unique binding site, also known as the idiotype of the T-cell clone. In contrast, the C domain does not participate in antigen binding.

A TCR is a heterodimeric cell surface protein of the immunoglobulin super-family, which is associated with invariant proteins of the CD3 complex involved in mediating signal transduction. TCRs exist in αβ and γδ forms, which are structurally similar but have quite distinct anatomical locations and probably functions. The extracellular portion of native heterodimeric αβTCR and γδTCR each contain two polypeptides, each of which has a membrane-proximal constant region, and a membrane-distal variable region. Each of the constant and variable regions include an intra-chain disulfide bond. The variable regions contain the highly polymorphic loops analogous to the complementarity determining regions (CDRs), also known as hypervariable regions, of antibodies. The variable regions of both the TCRα and TCRβ chain each have three CDRs, numbered CDR1, CDR2, and CDR3 in the direction from the amino terminal end to the carboxy terminal end. CDR3 is the main CDR responsible for recognizing processed antigen. The TCRβ CDR3 has been recognized as more structurally diverse than the other CDRs.

The techniques for determining CDRs are generally known in the art. In some embodiments, the CDRs can be determined by approaches based on cross-species sequence variability. In some embodiments, the CDRs can be determined by approaches based on crystallographic studies of antigen-antibody complexes. In addition, combinations of these approaches are sometimes used in the art to determine CDRs. In certain embodiments, CDRs can be determined using sequence-based prediction tools. Such tools are generally available in the art, e.g., the Loupe V(D)J Browser provided by 10× Genomics® (Pleasanton, Calif.). For instance, in one embodiment, the single cell TCR sequencing of epitope reactive T-cell population can be conducted using the 10× Genomics® platform. Then the sequence can be processed using the Loupe V(D)J Browser to identify the clonotypes, V(D)J genes, and the CDR motifs, etc. More detailed information about the Loupe V(D)J Browser is available over the world-wide-web at site: support.10×genomics.com/single-cell-vdj/software/visualization/latest/tutorial-clonotypes, which is herein incorporated by reference in its entirety.

### Chimeric Antigen Receptors

Chimeric antigen receptors (CARs) are engineered transmembrane chimeric proteins designed to assign antigen specificity to T-cells. They are recombinant receptors comprising an antigen binding region, a transmembrane region and an intracellular signaling region.

In certain embodiments, the CAR comprises one or more co-stimulatory domains comprising: CD28, ICOS, OX-40 or 41BB. The intracellular signaling region of a CAR or cell of the disclosure may comprise signaling regions from one, two, three, four or all five of these proteins in addition to the other regions specified herein.

The co-stimulatory domains of a CAR or cell of the disclosure may comprise co-stimulatory domains from both 41BB and CD28. The 41BB co-stimulatory domain can be downstream of the CD28 co-stimulatory domains.

The CAR may also comprise a spacer or hinge region situated between the antigen binding region and T cell plasma membrane. Commonly a spacer or hinge is a sequence derived from IgG subclass IgG1, IgG4, IgD or CD8. In certain embodiments, the hinge region comprises a CD28 motif. The hinge region can have any length. In some embodiments, the hinge region comprises 1 amino acid or 10 amino acids or 20 amino acids or 50 amino acids or 60 amino acids or 70 amino acids or 80 amino acids or 100 amino acids or 120 amino acids or 140 amino acids or 160 amino acids or 180 amino acids or 200 amino acids or 250 amino acids or 300 amino acids or any number therebetween.

A CAR may further comprise a linker region. This may be rich in glycine for flexibility. The linker region may be rich in serine and threonine for solubility. The linker region can connect to N-terminus of variable heavy (V_{H}) chain with the C-terminus of the variable light (V_{L}) chain or vice versa.

The CARs can be encoded by a vector and/or encompassed in one or more delivery vehicles and formulations as described in detail below.

Other methods known in the art include, but are not limited to, lentiviral transduction, transposon-based systems, direct RNA transfection, and CRISPR/Cas systems (e.g., type I, type II, or type III systems using a suitable Cas protein such Cas3, Cas4, Cas5, Cas5e (or CasD), Cas6, Cas6e, Cas6f, Cas7, Cas8a1, Cas8a2, Cas8b, Cas8c, Cas9, Cas10, Casl Od, CasF, CasG, CasH, Csy1, Csy2, Csy3, Cse1 (or CasA), Cse2 (or CasB), Cse3 (or CasE), Cse4 (or CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csz1, Csx15, Csf1, Csf2, Csf3, Csf4, and Cu1966, etc.).

Vectors can include, for example, origins of replication, scaffold attachment regions (SARs), and/or markers. A marker gene can confer a selectable phenotype on a host cell. For example, a marker can confer biocide resistance, such as resistance to an antibiotic (e.g., kanamycin, G418, bleomycin, or hygromycin). An expression vector can include a tag sequence designed to facilitate manipulation or detection (e.g., purification or localization) of the expressed polypeptide. Tag sequences, such as green fluorescent protein (GFP), glutathione S-transferase (GST), polyhistidine, c-myc, hemagglutinin, or FLAG™ tag (Kodak, New Haven, Conn.) sequences typically are expressed as a fusion with the encoded polypeptide. Such tags can be inserted anywhere within the polypeptide, including at either the carboxyl or amino terminus.

Additional expression vectors also can include, for example, segments of chromosomal, non-chromosomal and synthetic DNA sequences. Suitable vectors include derivatives of SV40 and known bacterial plasmids, e.g., E. coli plasmids col E1, pCR1, pBR322, pMal-C2, pET, pGEX, pMB9 and their derivatives, plasmids such as RP4; phage DNAs, e.g., the numerous derivatives of phage 1, e.g., NM989, and other phage DNA, e.g., M13 and filamentous single stranded phage DNA; yeast plasmids such as the 2μ plasmid or derivatives thereof, vectors useful in eukaryotic cells, such as vectors useful in insect or mammalian cells; vectors derived from combinations of plasmids and phage DNAs, such as plasmids that have been modified to employ phage DNA or other expression control sequences.

Several delivery methods may be utilized in conjunction with the isolated nucleic acid sequences for in vitro (cell cultures) and in vivo (animals and patients) systems. In one embodiment, a lentiviral gene delivery system may be utilized. Such a system offers stable, long term presence of the gene in dividing and non-dividing cells with broad tropism and the capacity for large DNA inserts. (Dull et al, J Virol, 72:8463-8471 1998). In an embodiment, adeno-associated virus (AAV) may be utilized as a delivery method. AAV is a non-pathogenic, single-stranded DNA virus that has been actively employed in recent years for delivering therapeutic gene in in vitro and in vivo systems (Choi et al, Curr Gene Ther, 5:299-310, 2005). AAV include serotypes 1 through 9. An example of non-viral delivery method may utilize nanoparticle technology. This platform has demonstrated utility as a pharmaceutical in vivo. Nanotechnology has improved transcytosis of drugs across tight epithelial and endothelial barriers. It offers targeted delivery of its payload to cells and tissues in a specific manner (Allen and Cullis, Science, 303:1818-1822, 1998).

The vector can also include a regulatory region. The term “regulatory region” refers to nucleotide sequences that influence transcription or translation initiation and rate, and stability and/or mobility of a transcription or translation product. Regulatory regions include, without limitation, promoter sequences, enhancer sequences, response elements, protein recognition sites, inducible elements, protein binding sequences, 5′ and 3′ untranslated regions (UTRs), transcriptional start sites, termination sequences, polyadenylation sequences, nuclear localization signals, and introns.

The term “operably linked” refers to positioning of a regulatory region and a sequence to be transcribed in a nucleic acid so as to influence transcription or translation of such a sequence. For example, to bring a coding sequence under the control of a promoter, the translation initiation site of the translational reading frame of the polypeptide is typically positioned between one and about fifty nucleotides downstream of the promoter. A promoter can, however, be positioned as much as about 5,000 nucleotides upstream of the translation initiation site or about 2,000 nucleotides upstream of the transcription start site. A promoter typically comprises at least a core (basal) promoter. A promoter also may include at least one control element, such as an enhancer sequence, an upstream element or an upstream activation region (UAR). The choice of promoters to be included depends upon several factors, including, but not limited to, efficiency, selectability, inducibility, desired expression level, and cell- or tissue-preferential expression. It is a routine matter for one of skill in the art to modulate the expression of a coding sequence by appropriately selecting and positioning promoters and other regulatory regions relative to the coding sequence.

Vectors include, for example, viral vectors (such as adenoviruses Ad, AAV, lentivirus, and vesicular stomatitis virus (VSV) and retroviruses), liposomes and other lipid-containing complexes, and other macromolecular complexes capable of mediating delivery of a polynucleotide to a host cell. Vectors can also comprise other components or functionalities that further modulate gene delivery and/or gene expression, or that otherwise provide beneficial properties to the targeted cells. As described and illustrated in more detail below, such other components include, for example, components that influence binding or targeting to cells (including components that mediate cell-type or tissue-specific binding); components that influence uptake of the vector nucleic acid by the cell; components that influence localization of the polynucleotide within the cell after uptake (such as agents mediating nuclear localization); and components that influence expression of the polynucleotide. Such components also might include markers, such as detectable and/or selectable markers that can be used to detect or select for cells that have taken up and are expressing the nucleic acid delivered by the vector. Such components can be provided as a natural feature of the vector (such as the use of certain viral vectors which have components or functionalities mediating binding and uptake), or vectors can be modified to provide such functionalities. Other vectors include those described by Chen et al.; Bio Techniques, 34: 167-171 (2003). A large variety of such vectors are known in the art and are generally available. A “recombinant viral vector” refers to a viral vector comprising one or more heterologous gene products or sequences. Since many viral vectors exhibit size-constraints associated with packaging, the heterologous gene products or sequences are typically introduced by replacing one or more portions of the viral genome. Such viruses may become replication-defective, requiring the deleted function(s) to be provided in trans during viral replication and encapsidation (by using, e.g., a helper virus or a packaging cell line carrying gene products necessary for replication and/or encapsidation). Modified viral vectors in which a polynucleotide to be delivered is carried on the outside of the viral particle have also been described (see, e.g., Curiel, D. T., et al. PNAS 88: 8850-8854, 1991).

Additional vectors include viral vectors, fusion proteins and chemical conjugates. Retroviral vectors include Moloney murine leukemia viruses and HIV-based viruses. One HIV based viral vector comprises at least two vectors wherein the gag and pol genes are from an HIV genome and the env gene is from another virus. DNA viral vectors include pox vectors such as orthopox or avipox vectors, herpesvirus vectors such as a herpes simplex I virus (HSV) vector [Geller, A. I. et al., J. Neurochem, 64: 487 (1995); Lim, F., et al., in DNA Cloning: Mammalian Systems, D. Glover, Ed. (Oxford Univ. Press, Oxford England) (1995); Geller, A. I. et al., Proc Natl. Acad. Sci.: U.S.A.:90 7603 (1993); Geller, A. I., et al., Proc Natl. Acad. Sci USA: 87:1149 (1990)], Adenovirus Vectors [LeGal LaSalle et al., Science, 259:988 (1993); Davidson, et al., Nat. Genet. 3: 219 (1993); Yang, et al., J. Virol. 69: 2004 (1995)] and Adeno-associated Virus Vectors [Kaplitt, M. G., et al., Nat. Genet. 8:148 (1994)].

The polynucleotides embodied herein may be used with a microdelivery vehicle such as cationic liposomes and adenoviral vectors. For a review of the procedures for liposome preparation, targeting and delivery of contents, see Mannino and Gould-Fogerite, Bio Techniques, 6:682 (1988). See also, Felgner and Holm, Bethesda Res. Lab. Focus, 11(2):21 (1989) and Maurer, R. A., Bethesda Res. Lab. Focus, 11(2):25 (1989).

Replication-defective recombinant adenoviral vectors, can be produced in accordance with known techniques. See, Quantin, et al., Proc. Natl. Acad. Sci. USA, 89:2581-2584 (1992); Stratford-Perricadet, et al., J. Clin. Invest., 90:626-630 (1992); and Rosenfeld, et al., Cell, 68:143-155 (1992).

Another method is to use single stranded DNA producing vectors which can produce the expressed products intracellularly. See for example, Chen et al, BioTechniques, 34: 167-171 (2003), which is incorporated herein, by reference, in its entirety.

The nucleic acid sequences of the disclosure can be delivered to an appropriate cell of a subject. This can be achieved by, for example, the use of a polymeric, biodegradable microparticle or microcapsule delivery vehicle, sized to optimize phagocytosis by phagocytic cells such as macrophages. For example, PLGA (poly-lacto-co-glycolide) microparticles approximately 1-10 μm in diameter can be used. The polynucleotide is encapsulated in these microparticles, which are taken up by macrophages and gradually biodegraded within the cell, thereby releasing the polynucleotide. Once released, the DNA is expressed within the cell. A second type of microparticle is intended not to be taken up directly by cells, but rather to serve primarily as a slow-release reservoir of nucleic acid that is taken up by cells only upon release from the micro-particle through biodegradation. These polymeric particles should therefore be large enough to preclude phagocytosis (i.e., larger than 5 μm and preferably larger than 20 μm). Another way to achieve uptake of the nucleic acid is using liposomes, prepared by standard methods. The nucleic acids can be incorporated alone into these delivery vehicles or co-incorporated with cell- or tissue-specific antibodies, for example, specific for Treg cells or delivery to tumor cells as a target. Alternatively, one can prepare a molecular complex composed of a plasmid or other vector attached to poly-L-lysine by electrostatic or covalent forces. Poly-L-lysine binds to a ligand that can bind to a receptor on target cells. Delivery of “naked DNA” (i.e., without a delivery vehicle) to an intramuscular, intradermal, or subcutaneous site, is another means to achieve in vivo expression. In the relevant polynucleotides (e.g., expression vectors) the nucleic acid sequence encoding an isolated nucleic acid sequence comprising a sequence encoding a CAR, as described above.

In some embodiments, the compositions of the disclosure can be formulated as a nanoparticle, for example, nanoparticles comprised of a core of high molecular weight linear polyethylenimine (LPEI) complexed with DNA and surrounded by a shell of polyethyleneglycol modified (PEGylated) low molecular weight LPEI. The nucleic acids and vectors may also be applied to a surface of a device (e.g., a catheter) or contained within a pump, patch, or other drug delivery device. The nucleic acids and vectors disclosed herein can be administered alone, or in a mixture, in the presence of a pharmaceutically acceptable excipient or carrier (e.g., physiological saline). The excipient or carrier is selected on the basis of the mode and route of administration. Suitable pharmaceutical carriers, as well as pharmaceutical necessities for use in pharmaceutical formulations, are described in Remington's Pharmaceutical Sciences (E. W. Martin), a well-known reference text in this field, and in the USP/NF (United States Pharmacopeia and the National Formulary).

In some embodiments, the compositions can be formulated as a nanoparticle encapsulating the compositions embodied herein.

Regardless of whether compositions are administered as nucleic acids or polypeptides, they are formulated in such a way as to promote uptake by the mammalian cell. Useful vector systems and formulations are described above. In some embodiments the vector can deliver the compositions to a specific cell type. The disclosure is not so limited however, and other methods of DNA delivery such as chemical transfection, using, for example calcium phosphate, DEAE dextran, liposomes, lipoplexes, surfactants, and perfluoro chemical liquids are also contemplated, as are physical delivery methods, such as electroporation, micro injection, ballistic particles, and “gene gun” systems.

### Antigen Binding Domain

Numerous antigen-binding domains are known in the art, including those based on the antigen binding site of an antibody, antibody mimetics, nanobodies, and T-cell receptor fragments. For example, the antigen-binding domain may comprise: a single-chain variable fragment (scFv) derived from a monoclonal antibody; a natural ligand of the target antigen; a peptide with sufficient affinity for the target; a single domain binder such as a camelid; an artificial binder single as a Darpin; or a single-chain derived from a T-cell receptor. Accordingly, the antigen specific binding domain includes, without limitation, an antibody, a T cell receptor fragment, a soluble T cell receptor, nanobody, aptamer, syn/notch recognition domain/effector domain pair, receptors, fragments or combinations thereof. In certain embodiments, the antigen specific binding domain is a T cell variable region fragments. In other embodiments, the antigen specific binding domain is an antibody or fragment thereof. The CAR can include single chains of T cell receptors and antibodies. In certain embodiments, the antigen binding domain is a single chain fragment is a single chain variable fragment (scFv).

In certain embodiments, the antigen binding domain is or comprises an antibody or antibody fragment, aptamers, proteins and the like. In certain embodiments, the antibodies are human antibodies, including any known to bind a targeting molecule. The term “antibody” herein is used in the broadest sense and includes polyclonal and monoclonal antibodies, including intact antibodies and functional (antigen-binding) antibody fragments, including fragment antigen binding (Fab) fragments, F(ab′)₂ fragments, Fab′ fragments, Fv fragments, recombinant IgG (rIgG) fragments, variable heavy chain (V_{H}) regions capable of specifically binding the antigen, single chain antibody fragments, including single chain variable fragments (scFv), and single domain antibodies (e.g., sdAb, sdFv, nanobody) fragments. The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific, antibodies, diabodies, triabodies, and tetrabodies, tandem di-scFv, tandem tri-scFv. Unless otherwise stated, the term “antibody” should be understood to encompass functional antibody fragments thereof. The term also encompasses intact or full-length antibodies, including antibodies of any class or sub-class, including IgG and sub-classes thereof, IgM, IgE, IgA, and IgD.

In some embodiments, the antigen-binding domain is a humanized antibody of fragments thereof. A “humanized” antibody is an antibody in which all or substantially all CDR amino acid residues are derived from non-human CDRs and all or substantially all FR amino acid residues are derived from human FRs. A humanized antibody optionally may include at least a portion of an antibody constant region derived from a human antibody. A “humanized form” of a non-human antibody, refers to a variant of the non-human antibody that has undergone humanization, typically to reduce immunogenicity to humans, while retaining the specificity and affinity of the parental non-human antibody. In some embodiments, some FR residues in a humanized antibody are substituted with corresponding residues from a non-human antibody (e.g., the antibody from which the CDR residues are derived), e.g., to restore or improve antibody specificity or affinity

In some embodiments, the heavy and light chains of an antibody can be full-length or can be an antigen-binding portion (a Fab, F(ab′)2, Fv or a single chain Fv fragment (scFv)). In other embodiments, the antibody heavy chain constant region is chosen from, e.g., IgG1, IgG2, IgG3, IgG4, IgM, IgA1, IgA2, IgD, and IgE, particularly chosen from, e.g., IgG1, IgG2, IgG3, and IgG4, more particularly, IgG1 (e.g., human IgG1). In another embodiment, the antibody light chain constant region is chosen from, e.g., kappa or lambda, particularly kappa.

Among the provided antibodies are antibody fragments. An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; variable heavy chain (Vii) regions, single-chain antibody molecules such as scFvs and single-domain V_{H} single antibodies; and multispecific antibodies formed from antibody fragments. In particular embodiments, the antibodies are single-chain antibody fragments comprising a variable heavy chain region and/or a variable light chain region, such as scFvs.

The term “variable region” or “variable domain”, when used in reference to an antibody, such as an antibody fragment, refers to the domain of an antibody heavy or light chain that is involved in binding the antibody to antigen. The variable domains of the heavy chain and light chain (V_{H} and V_{L}, respectively) of a native antibody generally have similar structures, with each domain comprising four conserved framework regions (FRs) and three CDRs. (See, e.g., Kindt et al. Kuby Immunology, 6th ed., W.H. Freeman and Co., page 91 (2007). A single V_{H} or V_{L} domain may be sufficient to confer antigen-binding specificity. Furthermore, antibodies that bind a particular antigen may be isolated using a V_{H} or V_{L} domain from an antibody that binds the antigen to screen a library of complementary V_{L} or V_{H} domains, respectively. See, e.g., Portolano et al., J. Immunol. 150:880-887 (1993); Clarkson et al., Nature 352:624-628 (1991).

Single-domain antibodies are antibody fragments comprising all or a portion of the heavy chain variable domain or all or a portion of the light chain variable domain of an antibody. In certain embodiments, a single-domain antibody is a human single-domain antibody.

Antibody fragments can be made by various techniques, including but not limited to proteolytic digestion of an intact antibody as well as production by recombinant host cells. In some embodiments, the antibodies are recombinantly-produced fragments, such as fragments comprising arrangements that do not occur naturally, such as those with two or more antibody regions or chains joined by synthetic linkers, e.g., peptide linkers, and/or that are may not be produced by enzyme digestion of a naturally-occurring intact antibody. In some aspects, the antibody fragments are scFvs.

In certain embodiments, the antibody or antibody fragments of the CAR have high binding affinity for a specific target antigen or post-translationally modified target antigens. In embodiments, the increased binding affinity is greater than effected by a reference antigen.

### Extracellular Matrix Proteins

A substantial portion of the volume of tissues is extracellular space, which is largely filled by an intricate network of macromolecules constituting the extracellular matrix (ECM). The ECM is composed of two major classes of biomolecules: glycosaminoglycans (GAGs), most often covalently linked to protein forming the proteoglycans, and fibrous proteins which include collagen, elastin, fibronectin, and laminin. These components are secreted locally and assembled into the organized meshwork that is the ECM (Alberts B, Johnson A, Lewis J, et al. Molecular Biology of the Cell. 4^{th} edition. New York: Garland Science; 2002. The Extracellular Matrix of Animals; Alexandra Naba, et al. “The extracellular matrix: Tools and insights for the “omics” era”, Matrix Biology, Volume 49, 2016, Pages 10-24, ISSN 0945-053X).

Connective tissue refers to the matrix composed of the ECM, cells (primarily fibroblasts), and ground substance that is tasked with holding other tissues and cells together forming the organs. Ground substance is a complex mixture of GAGs, proteoglycans, and glycoproteins (primarily laminin and fibronectin) but generally does not include the collagens. In most connective tissues, the matrix constituents are secreted principally by fibroblasts but in certain specialized types of connective tissues, such as cartilage and bone, these components are secreted by chondroblasts and osteoblasts, respectively. In addition to the extracellular matrix, typical connective tissues contain cells (primarily fibroblasts) all of which are surrounded by ground substance. The ECM is not only critical for connecting cells together to form the tissues, but is also a substrate upon which cell migration is guided during the process of embryonic development and importantly, during wound healing. In addition, the ECM is responsible for the relay of environmental signals to the surfaces of individual cells (Hynes R O, Naba A. Overview of the matrisome—an inventory of extracellular matrix constituents and functions. Cold Spring Marti Perspect Biol. 2.012; 4(1):a004903. Published 2012 Jan. 1. doi:10.1101/cshperspect.a004903).

The extracellular matrix is composed of three major classes of biomolecules:

Structural proteins: e.g. the collagen, the fibrillins, and elastin;

Specialized proteins: e.g. fibronectin, the various laminins, and the various integrins; and,

Proteoglycans: these are composed of a protein core to which is attached long chains of repeating disaccharide units termed of glycosaminoglycans (GAGs) forming extremely complex high molecular weight components of the ECM.

The core constituents of ECMs, e.g., collagens, elastin, fibronectin, laminins, proteoglycans (PGs), hyaluronan (HA), and several glycoproteins such as matricellular proteins, interact with each other creating a multicomponent structural meshwork that hosts several cell types. ECM molecules also interact with resident cells through numerous cell surface receptors. Classical ECM receptors include integrins, discoidin domain receptors (DDRs), cell surface PGs, HA receptors such as CD44, RHAMM, LYVE-1, and layilin. Moreover, matrix molecules interact and regulate signaling via other nonconventional receptors including growth factor receptors and toll-like receptors (TLRs). Various cell types including fibroblasts, immune cells, endothelial cells, epithelial cells, and pericytes that dwell within ECMs communicate via diverse cell surface receptors with matrix components to adjust their functions and behavior. Considering that ECMs undergo a continuous remodeling either under normal conditions such as wound healing or in pathological circumstances the maintenance of appropriate ECM composition and structure is of fundamental importance for tissue integrity and functionality. Usual matrix-degrading enzymes including matrix metalloproteinases (MMPs), a disintegrin and metalloproteinases (ADAMs), ADAMs with thrombospondin motifs (ADAMTS), plasminogen activators, as well as atypical proteases such as intracellular cathepsins and granzymes are involved in matrix remodeling. Other nonproteolytic enzymes including hyaluronidases and heparanase are implicated in the degradation of HA and glycosaminoglycans (GAGs) such as heparan sulfate (HS)/heparin (HP) chains present on PGs, respectively. Matrix degradation simultaneously coexists with the production and accumulation of newly formed matrix components. This fine-regulated process results in normal replacement of ECM. Although fibroblasts represent the major source of matrix components during matrix remodeling, all cells seem to contribute to the formation of matrix. Abnormal matrix remodeling is occurred in various pathologies and it constitutes a crucial factor for initiation or progression of several diseases (Achilleas D. et al. “The extracellular matrix as a multitasking player in disease”, The FEBS Journal (2019) doi:10.1111/febs.14818).

Although, fundamentally, the ECM is composed of water, proteins and polysaccharides, each tissue has an ECM with a unique composition and topology that is generated during tissue development through a dynamic and reciprocal, biochemical and biophysical dialogue between the various cellular components (e.g. epithelial, fibroblast, adipocyte, endothelial elements) and the evolving cellular and protein microenvironment. Indeed, the physical, topological, and biochemical composition of the ECM is not only tissue-specific, but is also markedly heterogeneous. Cell adhesion to the ECM is mediated by ECM receptors, such as integrins, discoidin domain receptors and syndecans (Frantz C, Stewart K M, Weaver V M. The extracellular matrix at a glance. J Cell Sci. 2010; 123(Pt 24):4195-4200. doi:10.1242/jcs.023820). Adhesion mediates cytoskeletal coupling to the ECM and is involved in cell migration through the ECM. Moreover, the ECM is a highly dynamic structure that is constantly being remodeled, either enzymatically or non-enzymatically, and its molecular components are subjected to a myriad of post-translational modifications. Through these physical and biochemical characteristics the ECM generates the biochemical and mechanical properties of each organ, such as its tensile and compressive strength and elasticity, and also mediates protection by a buffering action that maintains extracellular homeostasis and water retention. In addition, the ECM directs essential morphological organization and physiological function by binding growth factors (GFs) and interacting with cell-surface receptors to elicit signal transduction and regulate gene transcription. The biochemical and biomechanical, protective and organizational properties of the ECM in a given tissue can vary tremendously from one tissue to another (e.g. lungs versus skin versus bone) and even within one tissue (e.g. renal cortex versus renal medulla), as well as from one physiological state to another.

Accordingly, in certain embodiments, the CAR-T cells are specific for one or more extracellular matrix proteins, comprising collagens, laminins, fibronectins, tenascins, elastin, vitronectin, periostin, maltose-binding protein (MBP), glutamic acid decarboxylase 65-kilodalton protein (GAD65), gliadin, amyloid-beta (Aβ) peptide, Tau, TAR (transactive response) DNA-binding protein 43 (TDP-43), alpha-synuclein or combinations thereof. In certain embodiments, the extracellular matrix proteins or peptides thereof are modified. Examples of modification processes of extracellular matrix proteins or peptides thereof, comprise: acetylation, acylation, ADP-ribosylation, amidation, cross-linking, cyclization, disulfide bond formation, demethylation, formation of cysteine, formation of pyroglutamate, formylation, gamma-carboxylation, glycosylation, GPI anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins or combinations thereof.

### T Cells

Regulatory T cells (Tregs) are important in the maintenance of immune cell homeostasis as evidenced by the catastrophic consequences of genetic or physical ablation of the Treg population. Specifically, Treg cells maintain order in the immune system by enforcing a dominant negative regulation on other immune cells. Broadly classified into natural or adaptive (induced) Tregs; natural Tregs are CD4⁺CD25⁺ T-cells which develop, and emigrate from the thymus to perform their key role in immune homeostasis. Adaptive Tregs are non-regulatory CD4⁺ T-cells which acquire CD25 (IL-2R alpha) expression outside of the thymus, and are typically induced by inflammation and disease processes, such as autoimmunity and cancer.

There is increasing evidence that Tregs manifest their function through a myriad of mechanisms that include the secretion of immunosuppressive soluble factors such as IL-9, IL-10 and TGF beta, cell contact mediated regulation via the high affinity TCR and other costimulatory molecules such as CTLA-4, GITR, and cytolytic activity. Under the influence of TGF beta, adaptive Treg cells mature in peripheral sites, including mucosa-associated lymphoid tissue (MALT), from CD4⁺ Treg precursors, where they acquire the expression of markers typical of Tregs, including CD25, CTLA4 and GITR/AITR. Upon up-regulation of the transcription factor Foxp3, Treg cells begin their suppressive effect. This includes the secretion of cytokines including IL-10 and TGF beta which may induce cell-cycle arrest or apoptosis in effector T cells, and blocking co-stimulation and maturation of dendritic cells.

Tregs are hyporesponsive to TCR-mediated signaling, exhibiting low phosphorylation of CD3ζ, ERK, and AKT, among other downstream signaling molecules, when compared to Teff. Generating a CAR with a subdued TCR signal component could be beneficial for CAR Treg engineering. CD3ζ possesses three immunoreceptor tyrosine-based activation motifs (ITAMs), while other CD3 subunits, CD3γ, CD3δ, and CD3ε, possess one ITAM.

In addition to TCR-mediated signaling, in some embodiments, to promote full activation, a component for generating a secondary or co-stimulatory signal is also included. In some embodiments, the intracellular signaling domain comprises a CD28 co-stimulatory domain for Treg development, maintenance, and function. Absence of CD28 in Tregs does not affect Treg cell number; however, these cells have lower levels of CTLA-4, PD-1, and CCR6, and may result in systemic autoimmunity characterized by prominent skin inflammation. In NOD mice, CD28 deficiency may lead to defects in Treg development and homeostasis and exacerbated type 1 diabetes. Studies suggest that CD28 may function as an amplifier of TCR signaling; prolonged presence of antigen can sustain a functional T cell response in the absence of CD28 ((J. Holst et al., Scalable signaling mediated by T cell antigen receptor-CD3 ITAMs ensures effective negative selection and prevents autoimmunity. Nat Immunol 9, 658-666 (2008). R. Zhang, C. M. Borges, M. Y. Fan, J. E. Harris, L. A. Turka, Requirement for CD28 in Effector Regulatory T Cell Differentiation, CCR6 Induction, and Skin Homing. J Immunol 195, 4154-4161 (2015). B. Salomon et al., B7/CD28 costimulation is essential for the homeostasis of the CD4+CD25+ immunoregulatory T cells that control autoimmune diabetes. Immunity 12, 431-440 (2000). Q. Tang et al., Cutting edge: CD28 controls peripheral homeostasis of CD4+CD25+ regulatory T cells. J Immunol 171, 3348-3352 (2003)). CD28 contains a series of signaling motifs that can elicit intracellular phosphorylation cascades independent of TCR signals. CD28 tail motifs include an YMNM motif, which binds to the p85 subunit of PI3K, eliciting PI3K/Akt signaling, and a PYAP motif, which binds to FLNA, a regulator of cytoskeletal rearrangement, and the kinase LCK. In addition, both motifs bind the adaptor protein GRB2, which can bind Vav, which participates in various signaling complexes (J. S. Boomer, J. M. Green, An enigmatic tail of CD28 signaling. Cold Spring Harb Perspect Biol 2, a002436 (2010). A third motif present in the CD28 cytoplasmic domain is the PRRP motif, which binds the T cell-specific tyrosine kinase ITK and has been shown to be capable of inducing co-stimulation in murine primary T cells ((L. E. Marengere et al., The SH3 domain of Itk/Emt binds to proline-rich sequences in the cytoplasmic domain of the T cell costimulatory receptor CD28. J Immunol 159, 3220-3229 (1997). S. Ogawa et al., CD28 signaling in primary CD4(+) T cells: identification of both tyrosine phosphorylation-dependent and phosphorylation-independent pathways. Int Immunol 25, 671-681 (2013)).

As mentioned above, Tregs are hyporesponsive to TCR-mediated signaling when compared to Teff cells ((D. Yan, J. Farache, M. Mingueneau, D. Mathis, C. Benoist, Imbalanced signal transduction in regulatory T cells expressing the transcription factor FoxP3. Proc Natl Acad Sci USA 112, 14942-47 (2015). M. A. Gavin, S. R. Clarke, E. Negrou, A. Gallegos, A. Rudensky, Homeostasis and anergy of CD4(+)CD25(+) suppressor T cells in vivo. Nat Immunol 3, 33-41 (2002)). IL-2 signaling does not appear to trigger downstream targets of PI3K/Akt in Tregs, in contrast with Teff cells ((S. J. Bensinger et al., Distinct IL-2 receptor signaling pattern in CD4+CD25+ regulatory T cells. J Immunol 172, 5287-5296 (2004)).

Tregs are capable of constitutively expressing a range of receptors not found in Teff cells at steady state. These include the inhibitory receptors CTLA4, PD1, TIM3, LAG3, and TIGIT, whose presence in Teff cells may signify dysfunction or exhaustion ((A. C. Anderson, N. Joller, V. K. Kuchroo, Lag-3, Tim-3, and TIGIT: Co-inhibitory Receptors with Specialized Functions in Immune Regulation. Immunity 44, 989-1004 (2016). L. S. Walker, D. M. Sansom, Confusing signals: recent progress in CTLA-4 biology. Trends Immunol 36, 63-70 (2015)). Without wishing to be bound by theory, including signaling motifs from these molecules may produce CARs that work optimally in Tregs and maximize their suppressive function. Tregs are capable of upregulating the expression levels of several tumor necrosis factor receptor (TNFR) superfamily members upon maturation in vivo, such as 41BB, TACI, HVEM, GITR, OX40, CD27, CD30, and TNFR2 ((A. Vasanthakumar et al., The TNF Receptor Superfamily-NF-kappaB Axis Is Critical to Maintain Effector Regulatory T Cells in Lymphoid and Non-lymphoid Tissues. Cell Rep 20, 2906-2920 (2017). Y. Grinberg-Bleyer et al., Pathogenic T cells have a paradoxical protective effect in murine autoimmune diabetes by boosting Tregs. J Clin Invest 120, 4558-4568 (2010)).

### Isolation of Viable Treg Cells

In general, T regulatory cells were originally identified as a CD4⁺CD25⁺ T cell population with the capacity to suppress an immune response. The identification of Foxp3 as the “master-regulator” of Tregs was a critical step in defining Tregs as a distinct T cell lineage. The identification of additional antigenic markers on the surface of Tregs has enabled identification and FACS sorting of viable Tregs to greater purity, resulting in a more highly-enriched and suppressive Treg population. In addition to CD4 and CD25, it is now known that both mouse and human Tregs express GITR/AITR, CTLA-4, but express only low levels of CD127 (IL-7Ra). Moreover, Tregs can exist in different states which can be identified based on their expression of surface markers. Tregs which develop in the thymus from CD4⁺ thymocytes are known as “natural” Tregs, however Tregs can also be induced in the periphery from naïve CD4⁺ T cells in response to low-dose engagement of the TCR, TGF beta and IL-2. These “induced” Tregs secrete the immunosuppressive cytokine IL-10. The phenotype of Tregs changes again as they become activated, and markers including GARP in mouse and human, CD45RA in human, and CD103 in mouse have been shown to be useful for the identification of activated Tregs.

In certain embodiments, the T cell is a mammalian regulatory T cell (Treg), wherein the Treg cell is CD4⁺CD25⁺ CD127⁻, FOXP3⁺ and Helios⁺.

### Methods for Isolation of Cells

Any number of methods known in the art can be used to isolate cells, such as Tregs, or any other cell type that may be used in carrying out the treatment of a subject. Thus, also provided are various other genetically engineered cells expressing the chimeric antigen receptors e.g., CARs. The cells generally are eukaryotic cells, such as mammalian cells, and typically are human cells. In some embodiments, the cells are derived from the blood, bone marrow, lymph, or lymphoid organs, are cells of the immune system, such as cells of the innate or adaptive immunity, e.g., myeloid or lymphoid cells, including lymphocytes, typically T cells and/or NK cells. Other exemplary cells include stem cells, such as multipotent and pluripotent stem cells, including induced pluripotent stem cells (iPSCs). The cells typically are primary cells, such as those isolated directly from a subject and/or isolated from a subject and frozen. In some embodiments, the cells include one or more subsets of T cells or other cell types, such as whole T cell populations, CD4⁺ cells, CD8⁺ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. With reference to the subject to be treated, the cells may be allogeneic and/or autologous. Among the methods include off-the-shelf methods. In some aspects, such as for off-the-shelf technologies, the cells are pluripotent and/or multipotent, such as stem cells, such as induced pluripotent stem cells (iPSCs). In some embodiments, the methods include isolating cells from the subject, preparing, processing, culturing, and/or engineering them, as described herein, and re-introducing them into the same patient, before or after cryopreservation.

Among the sub-types and subpopulations of T cells and/or of CD4⁺ and/or of CD8⁺ T cells are naive T (T_{N}) cells, effector T cells (T_{EFF}), memory T cells and sub-types thereof, such as stem cell memory T (T_{SCMX} central memory T (T_{CM} effector memory T (T_{EM}), or terminally differentiated effector memory T cells, tumor-infiltrating lymphocytes (TIL), immature T cells, mature T cells, helper T cells, cytotoxic T cells, mucosa-associated invariant T (MATT) cells, naturally occurring and adaptive regulatory T (Treg) cells, helper T cells, such as T_{H}1 cells, T_{H}2 cells, T_{H}3 cells, T_{H}17 cells, T_{H}9 cells, T_{H}22 cells, follicular helper T cells, alpha/beta T cells, and delta/gamma T cells.

In some embodiments, the cells are natural killer (NK) cells. In some embodiments, the cells are monocytes or granulocytes, e.g., myeloid cells, macrophages, neutrophils, dendritic cells, mast cells, eosinophils, and/or basophils.

In some embodiments, the cells include one or more nucleic acids introduced via genetic engineering, and thereby express recombinant or genetically engineered products of such nucleic acids. In some embodiments, the nucleic acids are heterologous, i.e., normally not present in a cell or sample obtained from the cell, such as one obtained from another organism or cell, which for example, is not ordinarily found in the cell being engineered and/or an organism from which such cell is derived. In some embodiments, the nucleic acids are not naturally occurring, such as a nucleic acid not found in nature, including one comprising chimeric combinations of nucleic acids encoding various domains from multiple different cell types.

In some embodiments, preparation of the engineered cells includes one or more culture and/or preparation steps. The cells for introduction of the CAR, may be isolated from a sample, such as a biological sample, e.g., one obtained from or derived from a subject. In some embodiments, the subject from which the cell is isolated is one having the disease or condition or in need of a cell therapy or to which cell therapy will be administered. The subject in some embodiments is a human in need of a particular therapeutic intervention, such as the adoptive cell therapy for which cells are being isolated, processed, and/or engineered.

Accordingly, the cells in some embodiments are primary cells, e.g., primary human cells. The samples include tissue, fluid, and other samples taken directly from the subject, as well as samples resulting from one or more processing steps, such as separation, centrifugation, genetic engineering (e.g. transduction with viral vector), washing, and/or incubation. The biological sample can be a sample obtained directly from a biological source or a sample that is processed. Biological samples include, but are not limited to, body fluids, such as blood, plasma, serum, cerebrospinal fluid, synovial fluid, urine and sweat, tissue and organ samples, including processed samples derived therefrom.

In some aspects, the sample from which the cells are derived or isolated is blood or a blood-derived sample, or is or is derived from an apheresis or leukapheresis product. Exemplary samples include whole blood, peripheral blood mononuclear cells (PBMCs), leukocytes, bone marrow, thymus, tissue biopsy, tumor, leukemia, lymphoma, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom. Samples include, in the context of cell therapy, e.g., adoptive cell therapy, samples from autologous and allogeneic sources.

In some embodiments, the cells are derived from cell lines, e.g., T cell lines. The cells in some embodiments are obtained from a xenogeneic source, for example, from mouse, rat, non-human primate, or pig.

In some embodiments, isolation of the cells includes one or more preparation and/or non-affinity based cell separation steps. In some examples, cells are washed, centrifuged, and/or incubated in the presence of one or more reagents, for example, to remove unwanted components, enrich for desired components, lyse or remove cells sensitive to particular reagents. In some examples, cells are separated based on one or more property, such as density, adherent properties, size, sensitivity and/or resistance to particular components.

In some examples, cells from the circulating blood of a subject are obtained, e.g., by apheresis or leukapheresis. The samples, in some aspects, contain lymphocytes, including T cells, monocytes, granulocytes, B cells, other nucleated white blood cells, red blood cells, and/or platelets, and in some aspects contains cells other than red blood cells and platelets.

In some embodiments, the blood cells collected from the subject are washed, e.g., to remove the plasma fraction and to place the cells in an appropriate buffer or media for subsequent processing steps. In some embodiments, the cells are washed with phosphate buffered saline (PBS). In some embodiments, the wash solution lacks calcium and/or magnesium and/or many or all divalent cations. In some aspects, a washing step is accomplished a semi-automated “flow-through” centrifuge (for example, the Cobe 2991 cell processor, Baxter) according to the manufacturer's instructions. In some aspects, a washing step is accomplished by tangential flow filtration (TFF) according to the manufacturer's instructions. In some embodiments, the cells are resuspended in a variety of biocompatible buffers after washing, such as, for example, Ca⁺⁺/Mg⁺⁺ free PBS. In certain embodiments, components of a blood cell sample are removed and the cells directly resuspended in culture media.

In some embodiments, the methods include density-based cell separation methods, such as the preparation of white blood cells from peripheral blood by lysing the red blood cells and centrifugation through a Percoll or Ficoll gradient.

In some embodiments, the isolation methods include the separation of different cell types based on the expression or presence in the cell of one or more specific molecules, such as surface markers, e.g., surface proteins, intracellular markers, or nucleic acid. In some embodiments, any known method for separation based on such markers may be used. In some embodiments, the separation is affinity- or immunoaffinity-based separation. For example, the isolation in some aspects includes separation of cells and cell populations based on the cells' expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner.

Such separation steps can be based on positive selection, in which the cells having bound the reagents are retained for further use, and/or negative selection, in which the cells having not bound to the antibody or binding partner are retained. In some examples, both fractions are retained for further use. In some aspects, negative selection can be particularly useful where no antibody is available that specifically identifies a cell type in a heterogeneous population, such that separation is best carried out based on markers expressed by cells other than the desired population.

The separation need not result in 100% enrichment or removal of a particular cell population or cells expressing a particular marker. For example, positive selection of or enrichment for cells of a particular type, such as those expressing a marker, refers to increasing the number or percentage of such cells, but need not result in a complete absence of cells not expressing the marker. Likewise, negative selection, removal, or depletion of cells of a particular type, such as those expressing a marker, refers to decreasing the number or percentage of such cells, but need not result in a complete removal of all such cells.

In some examples, multiple rounds of separation steps are carried out, where the positively or negatively selected fraction from one step is subjected to another separation step, such as a subsequent positive or negative selection. In some examples, a single separation step can deplete cells expressing multiple markers simultaneously, such as by incubating cells with a plurality of antibodies or binding partners, each specific for a marker targeted for negative selection. Likewise, multiple cell types can simultaneously be positively selected by incubating cells with a plurality of antibodies or binding partners expressed on the various cell types. For example, in some aspects, specific subpopulations of T cells, such as cells positive or expressing one or more markers, e.g., CD4⁺CD25⁺, FOXP3⁺ and Helios⁺.

T cells, are isolated by positive or negative selection techniques. For example, CD3⁺, CD28⁺ T cells can be positively selected using anti-CD3/anti-CD28 conjugated magnetic beads (e.g., DYNABEADS® M-450 CD3/CD28 T Cell Expander).

In some embodiments, isolation is carried out by enrichment for a particular cell population by positive selection, or depletion of a particular cell population, by negative selection. In some embodiments, positive or negative selection is accomplished by incubating cells with one or more antibodies or other binding agent that specifically bind to one or more surface markers expressed or expressed (marker“1”) at a relatively higher level (marker^{high}) on the positively or negatively selected cells, respectively.

In some embodiments, T cells are separated from a PBMC sample by negative selection of markers expressed on non-T cells, such as B cells, monocytes, or other white blood cells, such as CD 14. In some aspects, a CD4⁺ or CD8⁺ selection step is used to separate CD4⁺ helper and CD8⁺ cytotoxic T cells. Such CD4⁺ and CD8⁺ populations can be further sorted into sub-populations by positive or negative selection for markers expressed or expressed to a relatively higher degree on one or more naive, memory, and/or effector T cell subpopulations.

In some embodiments, CD8⁺ cells are further enriched for or depleted of naïve, central memory, effector memory, and/or central memory stem cells, such as by positive or negative selection based on surface antigens associated with the respective subpopulation. In some embodiments, enrichment for central memory T (T_{CM}) cells is carried out to increase efficacy, such as to improve long-term survival, expansion, and/or engraftment following administration, which in some aspects is particularly robust in such sub-populations. See Terakura et al. (2012) Blood. 1:72-82; Wang et al. (2012) J Immunother. 35(9):689-701. In some embodiments, combining T_{CM}-enriched CD8⁺ T cells and CD4⁺ T cells further enhances efficacy.

In some embodiments, the enrichment for central memory T (T_{CM}) cells is based on positive or high surface expression of CD45RO, CD62L, CCR7, CD28, CD3, and/or CD 127; in some aspects, it is based on negative selection for cells expressing or highly expressing CD45RA and/or granzyme B.

In some aspects, a CD4 expression-based selection step is used to generate the CD4⁺ cell population or sub-population, such that both the positive and negative fractions from the CD4-based separation are retained and used in subsequent steps of the methods, optionally following one or more further positive or negative selection steps.

In one example, a sample of PBMCs or other white blood cell sample is subjected to selection of CD4⁺ cells, where both the negative and positive fractions are retained. The negative fraction then is subjected to negative selection based on expression o, for example, CD 14 and CD45RA, and positive selection based on a marker characteristic of central memory T cells, such as CD62L or CCR7, where the positive and negative selections are carried out in either order.

CD4⁺ T helper cells are sorted into naïve, central memory, and effector cells by identifying cell populations that have cell surface antigens. CD4⁺ lymphocytes can be obtained by standard methods. In some embodiments, naïve CD4⁺ T lymphocytes are CD45RO⁺, CD45RA⁺, CD62L⁺, CD4⁺ T cells. In some embodiments, central memory CD4⁺ cells are CD62L⁺ and CD45RO⁺.

In one example, to enrich for CD4⁺ cells by negative selection, a monoclonal antibody cocktail typically includes antibodies to CD14, CD20, CD11b, CD16, HLA-DR, and CD8. In some embodiments, the antibody or binding partner is bound to a solid support or matrix, such as a magnetic bead or paramagnetic bead, to allow for separation of cells for positive and/or negative selection. For example, in some embodiments, the cells and cell populations are separated or isolated using immunomagnetic (or affinitymagnetic) separation techniques (reviewed in Methods in Molecular Medicine, vol. 58: Metastasis Research Protocols, Vol. 2: Cell Behavior In Vitro and In Vivo, p 17-25 Edited by: S. A. Brooks and U. Schumacher © Humana Press Inc., Totowa, N.J.).

In some aspects, the sample or composition of cells to be separated is incubated with small, magnetizable or magnetically responsive material, such as magnetically responsive particles or microparticles, such as paramagnetic beads (e.g., such as DYNABEADS or MACS beads). The magnetically responsive material, e.g., particle, generally is directly or indirectly attached to a binding partner, e.g., an antibody, that specifically binds to a molecule, e.g., surface marker, present on the cell, cells, or population of cells that it is desired to separate, e.g., that it is desired to negatively or positively select.

In some embodiments, the magnetic particle or bead comprises a magnetically responsive material bound to a specific binding member, such as an antibody or other binding partner. There are many well-known magnetically responsive materials used in magnetic separation methods. Suitable magnetic particles include those described in Molday, U.S. Pat. No. 4,452,773, and in European Patent Specification EP 452342 B, which are hereby incorporated by reference. Colloidal sized particles, such as those described in Owen U.S. Pat. No. 4,795,698, and Liberti et al., U.S. Pat. No. 5,200,084 are other examples.

The incubation generally is carried out under conditions whereby the antibodies or binding partners, or molecules, such as secondary antibodies or other reagents, which specifically bind to such antibodies or binding partners, which are attached to the magnetic particle or bead, specifically bind to cell surface molecules if present on cells within the sample.

In some aspects, the sample is placed in a magnetic field, and those cells having magnetically responsive or magnetizable particles attached thereto will be attracted to the magnet and separated from the unlabeled cells. For positive selection, cells that are attracted to the magnet are retained; for negative selection, cells that are not attracted (unlabeled cells) are retained. In some aspects, a combination of positive and negative selection is performed during the same selection step, where the positive and negative fractions are retained and further processed or subject to further separation steps.

In certain embodiments, the magnetically responsive particles are coated in primary antibodies or other binding partners, secondary antibodies, lectins, enzymes, or streptavidin. In certain embodiments, the magnetic particles are attached to cells via a coating of primary antibodies specific for one or more markers. In certain embodiments, the cells, rather than the beads, are labeled with a primary antibody or binding partner, and then cell-type specific secondary antibody- or other binding partner (e.g., streptavidin)-coated magnetic particles, are added. In certain embodiments, streptavidin-coated magnetic particles are used in conjunction with biotinylated primary or secondary antibodies.

In some embodiments, the magnetically responsive particles are left attached to the cells that are to be subsequently incubated, cultured and/or engineered; in some aspects, the particles are left attached to the cells for administration to a patient. In some embodiments, the magnetizable or magnetically responsive particles are removed from the cells. Methods for removing magnetizable particles from cells are known and include, e.g., the use of competing non-labeled antibodies, magnetizable particles or antibodies conjugated to cleavable linkers, etc. In some embodiments, the magnetizable particles are biodegradable.

In some embodiments, the affinity-based selection is via magnetic-activated cell sorting (MACS) (Miltenyi Biotec, Auburn, Calif.). Magnetic Activated Cell Sorting (MACS) systems are capable of high-purity selection of cells having magnetized particles attached thereto. In certain embodiments, MACS operates in a mode wherein the non-target and target species are sequentially eluted after the application of the external magnetic field. That is, the cells attached to magnetized particles are held in place while the unattached species are eluted. Then, after this first elution step is completed, the species that were trapped in the magnetic field and were prevented from being eluted are freed in some manner such that they can be eluted and recovered. In certain embodiments, the non-target cells are labelled and depleted from the heterogeneous population of cells.

In certain embodiments, the isolation or separation is carried out using a system, device, or apparatus that carries out one or more of the isolation, cell preparation, separation, processing, incubation, culture, and/or formulation steps of the methods. In some aspects, the system is used to carry out each of these steps in a closed or sterile environment, for example, to minimize error, user handling and/or contamination. In one example, the system is a system as described in International Patent Application, Publication Nos. WO2009/072003, or US 20110003380 A1.

In some embodiments, the system or apparatus carries out one or more, e.g., all, of the isolation, processing, engineering, and formulation steps in an integrated or self-contained system, and/or in an automated or programmable fashion. In some aspects, the system or apparatus includes a computer and/or computer program in communication with the system or apparatus, which allows a user to program, control, assess the outcome of, and/or adjust various aspects of the processing, isolation, engineering, and formulation steps.

In some aspects, the separation and/or other steps is carried out using CliniMACS system (Miltenyi Biotec), for example, for automated separation of cells on a clinical-scale level in a closed and sterile system. Components can include an integrated microcomputer, magnetic separation unit, peristaltic pump, and various pinch valves. The integrated computer in some aspects controls all components of the instrument and directs the system to perform repeated procedures in a standardized sequence. The magnetic separation unit in some aspects includes a movable permanent magnet and a holder for the selection column. The peristaltic pump controls the flow rate throughout the tubing set and, together with the pinch valves, ensures the controlled flow of buffer through the system and continual suspension of cells.

The CliniMACS system in some aspects uses antibody-coupled magnetizable particles that are supplied in a sterile, non-pyrogenic solution. In some embodiments, after labelling of cells with magnetic particles the cells are washed to remove excess particles. A cell preparation bag is then connected to the tubing set, which in turn is connected to a bag containing buffer and a cell collection bag. The tubing set consists of pre-assembled sterile tubing, including a pre-column and a separation column, and are for single use only. After initiation of the separation program, the system automatically applies the cell sample onto the separation column. Labelled cells are retained within the column, while unlabeled cells are removed by a series of washing steps. In some embodiments, the cell populations for use with the methods described herein are unlabeled and are not retained in the column. In some embodiments, the cell populations for use with the methods described herein are labeled and are retained in the column. In some embodiments, the cell populations for use with the methods described herein are eluted from the column after removal of the magnetic field, and are collected within the cell collection bag.

In certain embodiments, separation and/or other steps are carried out using the CliniMACS Prodigy system (Miltenyi Biotec). The CliniMACS Prodigy system in some aspects is equipped with a cell processing unity that permits automated washing and fractionation of cells by centrifugation. The CliniMACS Prodigy system can also include an onboard camera and image recognition software that determines the optimal cell fractionation endpoint by discerning the macroscopic layers of the source cell product. For example, peripheral blood may be automatically separated into erythrocytes, white blood cells and plasma layers. The CliniMACS Prodigy system can also include an integrated cell cultivation chamber which accomplishes cell culture protocols such as, e.g., cell differentiation and expansion, antigen loading, and long-term cell culture. Input ports can allow for the sterile removal and replenishment of media and cells can be monitored using an integrated microscope. See, e.g., Klebanoff et al. (2012) J Immunother. 35(9): 651-660, Terakura et al. (2012) Blood. 1:72-82, and Wang et al. (2012) Immunother. 35(9):689-701.

In some embodiments, a cell population described herein is collected and enriched (or depleted) via flow cytometry, in which cells stained for multiple cell surface markers are carried in a fluidic stream. In some embodiments, a cell population described herein is collected and enriched (or depleted) via preparative scale (FACS)-sorting. In certain embodiments, a cell population described herein is collected and enriched (or depleted) by use of microelectromechanical systems (MEMS) chips in combination with a FACS-based detection system (see, e.g., WO 2010/033140, Cho et al. (2010) Lab Chip 10, 1567-1573; and Godin et al. (2008) J Biophoton. 1(5):355-376. In both cases, cells can be labeled with multiple markers, allowing for the isolation of well-defined T cell subsets at high purity.

In some embodiments, the antibodies or binding partners are labeled with one or more detectable marker, to facilitate separation for positive and/or negative selection. For example, separation may be based on binding to fluorescently labeled antibodies. In some examples, separation of cells based on binding of antibodies or other binding partners specific for one or more cell surface markers are carried in a fluidic stream, such as by fluorescence-activated cell sorting (FACS), including preparative scale (FACS) and/or microelectromechanical systems (MEMS) chips, e.g., in combination with a flow-cytometric detection system. Such methods allow for positive and negative selection based on multiple markers simultaneously.

In some embodiments, the preparation methods include steps for freezing, e.g., cryopreserving, the cells, either before or after isolation, incubation, and/or engineering. In some embodiments, the freeze and subsequent thaw step removes granulocytes and, to some extent, monocytes in the cell population. In some embodiments, the cells are suspended in a freezing solution, e.g., following a washing step to remove plasma and platelets. Any of a variety of known freezing solutions and parameters in some aspects may be used. One example involves using PBS containing 20% DMSO and 8% human serum albumin (HSA), or other suitable cell freezing media. This is then diluted 1:1 with media so that the final concentration of DMSO and HSA are 10% and 4%, respectively. The cells are then frozen to −80° C. at a rate of 1° per minute and stored in the vapor phase of a liquid nitrogen storage tank.

In some embodiments, the provided methods include cultivation, incubation, culture, and/or genetic engineering steps. For example, in some embodiments, provided are methods for incubating and/or engineering the depleted cell populations and culture-initiating compositions.

Thus, in some embodiments, the cell populations are incubated in a culture-initiating composition. The incubation and/or engineering may be carried out in a culture vessel, such as a unit, chamber, well, column, tube, tubing set, valve, vial, culture dish, bag, or other container for culture or cultivating cells.

In some embodiments, the cells are incubated and/or cultured prior to or in connection with genetic engineering. The incubation steps can include culture, cultivation, stimulation, activation, and/or propagation. In some embodiments, the compositions or cells are incubated in the presence of stimulating conditions or a stimulatory agent. Such conditions include those designed to induce proliferation, expansion, activation, and/or survival of cells in the population, to mimic antigen exposure, and/or to prime the cells for genetic engineering, such as for the introduction of a recombinant antigen receptor.

The conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells.

In some embodiments, the stimulating conditions or agents include one or more agent, e.g., ligand, which is capable of activating an intracellular signaling domain of a TCR complex. In some aspects, the agent turns on or initiates TCR/CD3 intracellular signaling cascade in a T cell. Such agents can include antibodies, such as those specific for a TCR, e.g. anti-CD3. In some embodiments, the stimulating conditions include one or more agent, e.g. ligand, which is capable of stimulating a costimulatory receptor, e.g., anti-CD28. In some embodiments, such agents and/or ligands may be, bound to solid support such as a bead, and/or one or more cytokines. Optionally, the expansion method may further comprise the step of adding anti-CD3 and/or anti CD28 antibody to the culture medium (e.g., at a concentration of at least about 0.5 ng/ml). In some embodiments, the stimulating agents include IL-2, IL-15 and/or IL-7. In some aspects, the IL-2 concentration is at least about 10 units/mL.

In some aspects, incubation is carried out in accordance with techniques such as those described in U.S. Pat. No. 6,040,177 to Riddell et al.; Klebanoff et al. (2012) J Immunother. 35(9): 651-660, Terakura et al. (2012) Blood. 1:72-82, and/or Wang et al. (2012) J Immunother. 35(9):689-701.

In some embodiments, the T cells are expanded by adding to the culture-initiating composition feeder cells, such as non-dividing peripheral blood mononuclear cells (PBMC), (e.g., such that the resulting population of cells contains at least about 5, 10, 20, or 40 or more PBMC feeder cells for each T lymphocyte in the initial population to be expanded); and incubating the culture (e.g. for a time sufficient to expand the numbers of T cells). In some aspects, the non-dividing feeder cells can comprise gamma-irradiated PBMC feeder cells. In some embodiments, the PBMC are irradiated with gamma rays in the range of about 3000 to 3600 rads to prevent cell division. In some aspects, the feeder cells are added to culture medium prior to the addition of the populations of T cells.

In some embodiments, the stimulating conditions include temperature suitable for the growth of human T lymphocytes, for example, at least about 25 degrees Celsius, generally at least about 30 degrees, and generally at or about 37 degrees Celsius. Optionally, the incubation may further comprise adding non-dividing EBV-transformed lymphoblastoid cells (LCL) as feeder cells. LCL can be irradiated with gamma rays in the range of about 6000 to 10,000 rads. The LCL feeder cells in some aspects is provided in any suitable amount, such as a ratio of LCL feeder cells to initial T lymphocytes of at least about 10:1.

### Methods of Treatment

In certain embodiments, a method of treating a subject diagnosed with an inflammatory disease comprises isolating T lymphocytes from a biological sample obtained from the subject; separating CD4⁺ T regulatory Cells (Treg) from conventional T cells (Tconv), wherein the Treg cells are CD4⁺CD25⁺CD127⁻ and the Tconv are CD4⁺CD25⁻CD127⁺; identifying modified protein or proteins at the site of the inflammation, transducing the Treg cells with an expression vector encoding a chimeric antigen receptor (CAR) which specifically binds to said modified protein or proteins; stimulating the transduced Treg with material obtain at the site of the inflamed lesion at least once ex vivo to obtain Treg cells specific for the modified protein or proteins; and reinfusing the Treg into the subject, thereby treating the subject. In certain embodiments, the Treg cells are autologous cells. CAR-T cells may be generated from any suitable source of T cells known in the art including, but not limited to, T cells collected from a subject. The subject may be a patient with an autoimmune disease such as rheumatoid arthritis, in need of CAR-T cell therapy or a subject of the same species as the subject with the autoimmune disease in need of CAR-T cell therapy. The collected T cells may be expanded ex vivo using methods commonly known in the art before transduction with a CAR to generate a CAR-T cell.

Methods for CAR design, delivery and expression in T cells, and the manufacturing of clinical-grade CAR-T cell populations are known in the art. See, for example, Lee et al., Clin. Cancer Res. 2012, 18(10): 2780-90, hereby incorporated by reference in its entirety. For example, the engineered CARs may be introduced into T cells using retroviruses, which efficiently and stably integrate a nucleic acid sequence encoding the chimeric antigen receptor into the target cell genome. The CAR-T cells, once they have been expanded ex vivo in response to, for example, an autoimmune disease antigen, can be reinfused into the subject in a therapeutically effective amount. The term “therapeutically effective amount” as used herein means the amount of CAR T cells when administered to a mammal, in particular a human, in need of such treatment, is sufficient to treat autoimmune diseases, or prevent organ rejection etc.

In certain embodiments, administration of any of the compositions embodied herein, e, can be combined with other cell-based therapies, for example, stem cells, antigen presenting cells, pancreatic islets etc.

The composition of the present disclosure may be prepared in a manner known in the art and in a manner suitable for parenteral administration to mammals, particularly humans, comprising a therapeutically effective amount of the composition alone, with one or more pharmaceutically acceptable carriers or diluents.

The term “pharmaceutically acceptable carrier” as used herein means any suitable carriers, diluents or excipients. These include all aqueous and non-aqueous isotonic sterile injection solutions which may contain anti-oxidants, buffers and solutes, which render the composition isotonic with the blood of the intended recipient; aqueous and non-aqueous sterile suspensions, which may include suspending agents and thickening agents, dispersion media, antifungal and antibacterial agents, isotonic and absorption agents and the like. It will be understood that compositions of the invention may also include other supplementary physiologically active agents.

The carrier must be pharmaceutically “acceptable” in the sense of being compatible with the other ingredients of the composition and not injurious to the subject. Compositions include those suitable for parenteral administration, including subcutaneous, intramuscular, intravenous and intradermal administration. The compositions may conveniently be presented in unit dosage form and may be prepared by any method well known in the art of pharmacy. Such methods include preparing the carrier for association with the CAR T cells. In general, the compositions are prepared by uniformly and intimately bringing into association any active ingredients with liquid carriers.

In an embodiment, the composition is suitable for parenteral administration. In another embodiment, the composition is suitable for intravenous administration.

Compositions suitable for parenteral administration include aqueous and nonaqueous isotonic sterile injection solutions which may contain anti-oxidants, buffers, bactericides and solutes, which render the composition isotonic with the blood of the intended recipient; and aqueous and non-aqueous sterile suspensions which may include suspending agents and thickening agents.

In other embodiments, the compositions comprise a cell which has been transformed or transfected with one or more vectors or nucleic acids encoding one or more CARs. In some embodiments, the methods of the disclosure can be applied ex vivo. That is, a subject's cells can be removed from the body and transduced with the compositions in culture with a desired target antigen, expand target-antigen specific, e.g. T cells and the expanded cells returned to the subject's body. The cell can be the subject's cells or they can be haplotype matched or a cell line. The cells can be irradiated to prevent replication. In some embodiments, the cells are human leukocyte antigen (HLA)-matched, autologous, cell lines, or combinations thereof. In other embodiments the cells can be a stem cell. For example, an embryonic stem cell or an artificial pluripotent stem cell (induced pluripotent stem cell (iPS cell)). Embryonic stem cells (ES cells) and artificial pluripotent stem cells (induced pluripotent stem cell, iPS cells) have been established from many animal species, including humans. These types of pluripotent stem cells would be the most useful source of cells for regenerative medicine because these cells are capable of differentiation into almost all of the organs by appropriate induction of their differentiation, with retaining their ability of actively dividing while maintaining their pluripotency. iPS cells, in particular, can be established from self-derived somatic cells, and therefore are not likely to cause ethical and social issues, in comparison with ES cells which are produced by destruction of embryos. Further, iPS cells, which are self-derived cell, make it possible to avoid rejection reactions, which are the biggest obstacle to regenerative medicine or transplantation therapy.

The CARs can be easily delivered to a subject by methods known in the art, for example, methods which deliver siRNA. Thus, the, CAR molecules can be used clinically, similar to the approaches taken by current gene therapy. In particular, a CAR stable expression stem cell or iPS cells for cell transplantation therapy as well as vaccination can be developed for use in subjects.

The CAR-T cells, once they have been expanded ex vivo in response to an autoimmune disease antigen, are reinfused into the subject in a therapeutically effective amount. The term “therapeutically effective amount” as used herein means the amount of CAR T cells when administered to a mammal, in particular a human, in need of such treatment, is sufficient to treat autoimmune diseases such as rheumatoid arthritis.

The precise amount of CAR T cells to be administered can be determined by a physician with consideration of individual differences in age, weight, extent of disease and condition of the subject.

Typically, administration of T cell therapies is defined by number of cells per kilogram of body weight. However, because T cells will replicate and expand after transfer, the administered cell dose will not resemble the final steady-state number of cells.

In an embodiment, a pharmaceutical composition comprising the CAR T cells of the present disclosure may be administered at a dosage of 10⁴ to 10⁹ cells/kg body weight. In another embodiment, a pharmaceutical composition comprising the CAR T cells of the present disclosure may be administered at a dosage of 10⁵ to 10⁶ cells/kg body weight, including all integer values within those ranges.

Compositions comprising the CAR T cells of the present disclosure may also be administered multiple times at these dosages. The cells can be administered by using infusion techniques that are known in the art (see, for example, Rosenberg et al., 1988, New England Journal of Medicine, 319: 1676). The optimal dosage and treatment regimen for a particular subject can be readily determined by one skilled in the art by monitoring the patient for signs of disease and adjusting the treatment accordingly.

In certain embodiments, administration of any of the compositions embodied herein, for the treatment of an autoimmune disease, can be combined with other cell-based therapies, for example, stem cells, antigen presenting cells, etc.

The composition of the present disclosure may be prepared in a manner known in the art and are those suitable for parenteral administration to mammals, particularly humans, comprising a therapeutically effective amount of the composition alone, with one or more pharmaceutically acceptable carriers or diluents.

The term “pharmaceutically acceptable carrier” as used herein means any suitable carriers, diluents or excipients. These include all aqueous and non-aqueous isotonic sterile injection solutions which may contain anti-oxidants, buffers and solutes, which render the composition isotonic with the blood of the intended recipient; aqueous and non-aqueous sterile suspensions, which may include suspending agents and thickening agents, dispersion media, antifungal and antibacterial agents, isotonic and absorption agents and the like. It will be understood that compositions of the disclosure may also include other supplementary physiologically active agents.

The carrier must be pharmaceutically “acceptable” in the sense of being compatible with the other ingredients of the composition and not injurious to the subject. Compositions include those suitable for parenteral administration, including subcutaneous, intramuscular, intravenous and intradermal administration. The compositions may conveniently be presented in unit dosage form and may be prepared by any method well known in the art of pharmacy. Such methods include preparing the carrier for association with the CAR T cells. In general, the compositions are prepared by uniformly and intimately bringing into association any active ingredients with liquid carriers.

In an embodiment, the composition is suitable for parenteral administration. In another embodiment, the composition is suitable for intravenous administration.

Compositions suitable for parenteral administration include aqueous and nonaqueous isotonic sterile injection solutions which may contain anti-oxidants, buffers, bactericides and solutes, which render the composition isotonic with the blood of the intended recipient; and aqueous and non-aqueous sterile suspensions which may include suspending agents and thickening agents.

The disclosure also contemplates the combination of the composition of the present disclosure with other drugs and/or in addition to other treatment regimens or modalities such as surgery. When the composition of the present disclosure is used in combination with known therapeutic agents the combination may be administered either in sequence (either continuously or broken up by periods of no treatment) or concurrently or as an admixture. In the case of autoimmune diseases, e.g. rheumatoid arthritis, treatment comprises administering to the subject the compositions embodied herein, e.g. autologous T cells transduced with CAR specific for modified proteins, peptides or fragments thereof derived from extracellular spaces at a site of an inflammation in a subject. In certain embodiments, the modified protein, peptide or fragments thereof are modified by one or more processes comprising: acetylation, acylation, ADP-ribosylation, amidation, cross-linking, cyclization, disulfide bond formation, demethylation, formation of cysteine, formation of pyroglutamate, formylation, gamma-carboxylation, glycosylation, GPI anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins or combinations thereof. In certain embodiments, the modified protein, peptide or fragments thereof are modified by attachment of one or more molecules comprising: a flavin, a heme moiety, a nucleotide, a nucleotide derivative, a lipid, a lipid derivative, a phosphotidylinositol, or combinations thereof. In certain embodiments, one or more anti-inflammatory agents and/or therapeutic agents are administered. The one or more anti-inflammatory agents and/or therapeutic agents may be administered simultaneously or sequentially. The anti-inflammatory agents comprise one or more antibodies which specifically bind to pro-inflammatory cytokines, e.g. pro-inflammatory cytokines such as IL-1, IL-4, IL-5, IL-6, IL-8, IL-10, GM-CSF, TNFα IFN-γ and MIP-1a. In certain embodiments, the antibodies are anti-TNFα, anti-IL-6 or combinations thereof. In certain embodiments, one or more agents, other than antibodies can be administered which decrease pro-inflammatory cytokines, e.g. non-steroidal anti-inflammatory drugs (NSAIDs). Any combination of antibodies and one or more agents can be administered which decrease pro-inflammatory cytokines.

In certain embodiments, the composition comprising CAR T cells is administered with an anti-inflammatory agent. Anti-inflammatory agents or drugs include, but are not limited to, steroids and glucocorticoids (including betamethasone, budesonide, dexamethasone, hydrocortisone acetate, hydrocortisone, hydrocortisone, methylprednisolone, prednisolone, prednisone, triamcinolone), nonsteroidal anti-inflammatory drugs (NSAIDS) including aspirin, ibuprofen, naproxen, methotrexate, sulfasalazine, leflunomide, anti-TNF medications, cyclophosphamide and mycophenolate.

Other exemplary NSAIDs are chosen from the group consisting of ibuprofen, naproxen, naproxen sodium, Cox-2 inhibitors such as VIOXX™ (rofecoxib) and CELEBREX™ (celecoxib), and sialylates. Exemplary analgesics are chosen from the group consisting of acetaminophen, oxycodone, tramadol of proporxyphene hydrochloride. Exemplary glucocorticoids are chosen from the group consisting of cortisone, dexamethasone, hydrocortisone, methylprednisolone, prednisolone, or prednisone. Exemplary biological response modifiers include molecules directed against cell surface markers (e.g., CD4, CD5, etc.), cytokine inhibitors, such as the TNF antagonists (e.g., etanercept (ENBREL™), adalimumab (HUMIRA™) and infliximab (REMICADE™), chemokine inhibitors and adhesion molecule inhibitors. The biological response modifiers include monoclonal antibodies as well as recombinant forms of molecules. Exemplary disease-modifying anti-rheumatic drugs (DMARDs) include azathioprine, cyclophosphamide, cyclosporine, methotrexate, penicillamine, leflunomide, sulfasalazine, hydroxychloroquine, Gold (oral (auranofin) and intramuscular) and minocycline.

In certain embodiments, the compositions contemplated herein are administered in conjunction with a cytokine. By “cytokine” as used herein is meant a generic term for proteins released by one cell population that act on another cell as intercellular mediators. Examples of such cytokines are lymphokines, monokines, chemokines, and traditional polypeptide hormones. Included among the cytokines are growth hormones such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; fibroblast growth factor; prolactin; placental lactogen; tumor necrosis factor-alpha and -beta; mullerian-inhibiting substance; mouse gonadotropin-associated peptide; inhibin; activin; vascular endothelial growth factor; integrin; thrombopoietin (TPO); nerve growth factors such as NGF-beta; platelet-growth factor; transforming growth factors (TGFs) such as TGF-alpha and TGF-beta; insulin-like growth factor-I and -II; erythropoietin (EPO); osteoinductive factors; interferons such as interferon-alpha, beta, and -gamma; colony stimulating factors (CSFs) such as macrophage-CSF (M-CSF); granulocyte-macrophage-CSF (GM-CSF); and granulocyte-CSF (G-CSF); interleukins (ILs) such as IL-1, IL-1alpha, IL-2, IL-3, IL-4, IL-5, IL-6, IL-7, IL-8, IL-9, IL-10, IL-11, IL-12; IL-15, a tumor necrosis factor such as TNF-alpha or TNF-beta; and other polypeptide factors including LIF and kit ligand (KL). As used herein, the term cytokine includes proteins from natural sources or from recombinant cell culture, and biologically active equivalents of the native sequence cytokines.

Treatment in combination is also contemplated to encompass the treatment with either the composition of the disclosure followed by a known treatment, or treatment with a known agent followed by treatment with the composition of the disclosure, for example, as maintenance therapy. For example, in the treatment of autoimmune diseases, excessive and prolonged activation of immune cells, such as T and B lymphocytes, and overexpression of the master pro-inflammatory cytokine tumor necrosis factor alpha (TNF), together with other mediators such as interlukin-6 (IL-6), interlukin-1 (IL-1), and interferon gamma (IFN-γ), play a central role in the pathogenesis of autoimmune inflammatory responses in rheumatoid arthritis (RA), inflammatory bowel disease (IBD), Crohn's disease (CD), and ankylosing spondylitis (AS).

Non-steroidal anti-inflammatory drugs (NSAIDs), glucocorticoids, disease-modifying anti-rheumatic drugs (DMARDs) are traditionally used in the treatment of autoimmune inflammatory diseases. NSAIDs and glucocorticoids are effective in the alleviation of pain and inhibition of inflammation, while DMARDs have the capacity of reducing tissue and organ damage caused by inflammatory responses. More recently, treatment for RA and other autoimmune diseases has been revolutionized with the discovery that TNF is critically important in the development of the diseases. Anti-TNF biologics (such as infliximab, adalimumab, etanercept, golimumab, and certolizumab pepol) have markedly improved the outcome of the management of autoimmune inflammatory diseases.

Non-steroidal anti-inflammatory drugs have analgesic, antipyretic, and anti-inflammatory effects, frequently used for the treatment of conditions like arthritis and headaches. NSAIDs relieve pain through blocking cyclooxygenase (COX) enzymes. COX promotes the production of prostaglandins, a mediator which causes inflammation and pain. Although NSAIDs have different chemical structures, all of them have the similar therapeutic effect, e.g., inhibition of autoimmune inflammatory responses. In general, NSAIDs can be divided into two broad categories: traditional non-selective NSAIDs and selective cyclooxygenase-2 (COX-2) inhibitors (For a review see, P. Li et al. Front Pharmacol. 2017; 8: 460).

In addition to anti-TNF agents, the biologics targeting other proinflammatory cytokines or immune competent molecules have also been extensively studied and actively developed. For example, abatacept, a fully humanized fusion protein of extracellular domain of CTLA-4 and Fc fraction of IgG1, has been approved for the RA patients with inadequate response to anti-TNF therapy. The major immunological mechanism of abatacept is selective inhibition of co-stimulation pathway (CD80 and CD86) and activation of T cells. Tocilizumab, a humanized anti-IL-6 receptor monoclonal antibody was approved for RA patients intolerant to DMARDs and/or anti-TNF biologics. This therapeutic mAb blocks the transmembrane signaling of IL-6 through binding with soluble and membrane forms of IL-6 receptor. Biological drugs targeting IL-1 (anakinra), Th1 immune responses (IL-12/IL-23, ustekinumab), Th17 immune responses (IL-17, secukinumab) and CD20 (rituximab) have also been approved for the treatment of autoimmune diseases (For a review see, P. Li et al. Front Pharmacol. 2017; 8: 460).

Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

### EXAMPLES

### Example 1: CAR-T Cells Specific for Extracellular Peptides and Modified Extracellular Peptides

Based on the possibility that rather than suppressing immunity, Tregs could destroy the tissue cells that are supposed to be protected led to the hypothesis that Tregs and other regulatory cell populations might not need to be directed at target tissue cells but rather the tissue microenvironment as a means of activating the cells in order to promote bystander suppression and infectious tolerance. In an initial proof of principle, it was shown that Tregs expressing a CAR that recognized citrullinated vimentin, a modified form of vimentin specifically found in the joints of patients with Rheumatoid Arthritis, can be triggered by synovial joint fluid from affects patients due to aggregation of the protein on the extracellular matrix. Moreover, the Tregs, once activated suppress T effector responses.

CAR Tregs as a cell therapeutic approach is to cure patients suffering of autoimmune or non-autoimmune inflammatory disorders by restoring their immune tolerance whereas the current drugs are administrated long-life after the disease onset and only slow down the progression of the disease but do not cure the patient.

Herein, it is proposed to expand the application of this technology for the treatment of a number of diseases. The basic concept is based on the identification of modified proteins present at the site of inflammation in the extracellular space either as a consequence of secretion, cell death or extracellular modifications. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins such as arginylation, and ubiquitination. (See, for instance, PROTEINS—STRUCTURE AND MOLECULAR PROPERTIES, 2nd Ed., T. E. Creighton, W. H. Freeman and Company, New York (1993); POSTTRANSLATIONAL COVALENT MODIFICATION OF PROTEINS, B. C. Johnson, Ed., Academic Press, New York, pgs. 1-12 (1983); Seifter et al., Meth Enzymol 182:626-646 (1990); Rattan et al., Ann NY Acad Sci 663:48-62 (1992).) Specific examples include: modifications such as amidation, citrullation and other modifications; denaturation as seen in the case of Tau, Amyloids and other prion-like proteins; and aggregated proteins generating novel epitopes recognized by a unique set of mAbs. Examples of disease applications include: Alzheimer's, Frontotemporal Dementias, Duchenne's Muscular Dystrophy, Parkinson's, COPD and many others.

Conferring specificity to a modified protein exclusively present at the site of the disease by engineering autologous CAR Tregs will avoid a systemic immunosuppression. Infused Tregs will be activated and perform their suppressive function at the site of the unwarranted inflammation. Most of the current drugs for patients suffering of autoimmune or non-autoimmune inflammatory disorder have systemic effects on the immune system rendering the patient more susceptible to severe infections and cancer and other off-target adverse effects.

Materials and Methods

The present disclosure comprises the following steps:

1) Identify modified protein or proteins present in the extracellular space of an inflammatory lesion in a subject.
2) Identify antibodies that recognizes an epitope present on the modified protein or proteins but not on unmodified proteins.
3) Generate a CAR that specifically binds to the modified protein or proteins.
4) Express the CAR of step 3 in a T reg or other regulatory cell to generate CAR-T cells. 5) Demonstrate specific activation of the CAR-T cells from step 4 with extracellular material taken from the inflamed lesion.
6) Use the activated CAR-T cells from step 5 to treat the subject of step 1.

Generation of Lentiviral Vectors Expressing CV-CAR.

Generation of CV-specific scFv: The variable regions of the heavy and light chain of the BVCA1 antibody were sequenced and used to generate CV-specific scFv gene in a V_{H}-linker-V_{L} format. The scFv protein was produced by inserting the sequence into a pSYN plasmid and inoculated into DH5-alpha E. coli competent cells. A single colony was grown in 5 ml of 2YT medium supplemented with 2% glucose and 100 μg/ml of ampicillin overnight at 30° C. in a shaker. The 5 ml of 0/N culture was inoculated into 500 ml fresh 2YT medium (with 0.1% glucose and 100 μg/ml of ampicillin) and incubated at 37° C. for 2.5 hours until OD₆₀₀=0.9. Expression of the scFv was induced by adding 250 μl of Isopropyl-β-d-thiogalactopyranoside (IPTG) and then incubate at 30° C. for 4 hrs with shaking. After 20 min of centrifugation at 5000 rpm, the bacterial pellet was re-suspended with 12.5 ml ice cold Periplasmic extraction buffer (PPB, 200 g/L Sucrose, 30 mM Tris-HCl, pH 8.0) and kept on ice 0/N. The next day bacteria were centrifuged at 10,000 rpm for 30 min, the supernatant was kept, and the pellet re-suspended with 12.5 ml of 5 mM ice cold Mg₂SO₄ and kept on ice for 30 min to induce an osmotic shock. The lysed bacteria were centrifuged at 10,000 rpm for 30 min and the supernatant was combined with the previous one. The CV-specific scFv was then purified by Ni-NTA chromatography.

Generation of CV-specific CAR constructs: By using Gibson assembly method (Gibson Assembly Master Mix, BioLabs, according to the manufacturer's instructions) the anti-CD19 scFv sequence was replaced by the CV-specific scFv sequence into already existing 19-CAR constructs cloned in p10001 lentiviral plasmid with rather CD28z or 41BBz intracellular domains followed by a CD3ζ domain and a truncated version of EGFR (EGFRt) used as a receptor and separated from the CAR by a T2A domain (CAR constructs were provided by Juno Therapeutics). These CAR constructs were both with a short hinge (IgG4—36 bp) so the Gibson assembly method was again used to replace the short hinge by a long hinge (117 bp of the extracellular domain of human CD28 sequence). Thus, multiple CV-specific CAR constructs were generated with a short or long hinge and 41BB or CD28 co-stimulatory domains.

HEK 293T cells transfection and lentivirus particles titration: HEK 293T cells were plated at 800,000 cells per well in a 6-well plate with 2 ml DMEM high glucose media supplemented with 10% FBS (without antibiotics) and placed at 37° C. 5% CO₂ overnight. At 80% of confluence in the wells, a mix of 1.5 ug of CV-CAR p10001 with 1.33 μg of packaging vector p8.91, 0.168 μg of VSV envelope vector pMD2.G and 9 μg of FuGENE HD Transfection Reagent (Promega) was gently added drop by drop to each well. To improve the efficiency of the viral particle production, the media was replaced after 14 hours by fresh 10% FBS-DMEM high glucose media supplemented with ViralBoost Reagent (diluted at 1/500; VC-100, Alstem). The virus supernatant was harvested 2 days later and concentrated 100 folds using a lentivirus precipitation solution (VP100, Alstem, manufacturer's instructions were followed). The transfection efficiency of the HEK 293T cells was assessed by flow cytometry using an anti-EGFRt Ab (Erbitux, Juno Therapeutics). To titer the lentiviral particles, Jurkat cells (10,000/well in 96-well plate) were place in culture with a serial dilution of virus for 4 days, then stained with anti-EGFRt Ab and analyzed by flow cytometry (LSR II; BD Biosciences). The dilutions yielding 1% to 20% of positive cells were used to determine the virus titer by following this equation: [number of target cells×(% of EGFRt⁺ cells/100)]/Volume of supernatant (ml).

CV-CAR construct generation: To generate a CV-CAR construct expressed in a lentiviral vector, a single-chain variable fragment (scFv) of a CD19-CAR construct present in a lentiviral backbone plasmid p10001 (available from Juno Therapeutics, Inc.) was replaced by a BVCA1 scFv using Gibson assembly method. As only a few differences are observed between the amino acid sequences of human and murine vimentin, the anti-CV antibody shows high specificity for both human and murine peptides. This allows performing the assessment both on human samples in vitro and on an RA mouse model in vivo. The dissociation constant (K_{D}) between the BVCA1 fully human IgG and the human CV peptide is about 10 nM, and the K_{D} between the BVCA1 scFv and the human CV peptide is about 198 nM. The results show that, as BVCA1 antibody, BVCA1 scFv is specific for citrullinated vimentin.

Two versions of the CV-specific CAR were created, each containing CD3ζ plus either CD28 (CV.28z-CAR) or 41BB (CV.41BBz-CAR) co-stimulatory domains. The CV-CAR construct in accordance with the described embodiments can have any suitable type(s) of co-stimulatory domains. A truncated version of EGFR gene separated from the CAR by a T2A peptide was used as a reporter gene. Lentiviral particles were produced by transfecting HEK 293 T cells. The supernatant was collected at day 3 and viral particles were precipitated to be enriched.

Assessment of CV-CAR construct hinges of different lengths: The constructs having hinges of two different lengths were compared: (1) a short hinge derived from IgG4 motif, and (2) a long hinge that is a portion of the extracellular domain of human CD28. It was observed that, for both CV.28z-CAR and CV.41BBz-CAR, the presence of the long hinge induced a more efficient activation of the CV-CAR T cells. Depending of the target antigen of the CAR construct, the optimal length of the hinge can be determined to allow a proper antigen-binding. To assess which length of hinge was optimal for the CV-specific CAR Treg activation, two different hinges were compared—a short hinge, IgG4 (36 bp) and a long hinge created using a portion of the extracellular domain of human CD28 (117 bp). To study these two different hinges in both CV-CAR with CD28z intracellular domain and CV-CAR with 41BBz intracellular domain, four versions of the CV-CAR construct were generated: two with the short hinge (CV-IgG4-28z and CV-IgG4-41BBz) and two with the long hinge (CV-CD28-28z and CV-CD28-41BBz). Expression of these different CV-CAR constructs into Tregs was induced by lentiviral transduction and the activation profile of the CV-CAR Tregs after being re-stimulated in presence CV-SA beads was analyzed. At day 3, a higher percentage of cells expressed the activation marker CD71 in the populations of CV-CAR Tregs expressing a CAR construct with the long hinge compared to the ones with the short hinge. The same observation can be made when assessing CD25 mean fluorescence intensity (MFI) at day 3. Moreover, the CV-CAR Tregs with the long hinge expanded more efficiently than the one with the short hinge.

Assessment of different versions of the scFv: The inventors developed a novel scFv, BVCA1, that binds specifically citrullinated vimentin protein. Most of the antibodies targeting citrullinated proteins found in RA patients are less specific and cross-react with a multitude of citrullinated proteins. Thus, the further analysis used BVCA1. However, from this single IgG, three versions of scFv were generated: (1) one with the V_{L} and V_{H} chains with the nucleotide sequence of origin with a 24 amino acid linker (scFv #1), (2) one with the nucleotide sequence of origin and a (GGGGS)₃ linker (scFv #2,) and (3) a third one with a codon-optimized sequence of V_{L} and V_{H} chains with a (GGGGS)₃ linker (scFv #3).

Only the CV-CAR with the scFv #1 was efficiently expressed at the cells surface of the Tregs and T cony at day 3 after transduction, whereas the expression of the reporter gene suggested a transduction efficacy comparable between the 3 different CAR constructs. At day 2 after re-stimulation in presence of CV-pep-SA beads (CV beads), a higher percentage of cells expressing the activation markers CD71 and CD69 in the Treg population previously transduced with the CV-CAR scFv #1 was observed, compared with the two other groups of CV-CAR Tregs. Based on these results, the CV-CAR construct with the scFv #1 was selected to continue the development of the CV-CAR constructs.

Generation of TCR-knock-out CV-CAR⁺ Treg cells using CRISPR/Cas9 technology. Treg cells were isolated from fresh PBMCs by flow cytometry as described in Example 3, centrifuged for 10 min at 90 g and resuspended in Lonza electroporation buffer P3 using 20 μl buffer per 1 million cells. Treg cells were then electroporated with CRISPR-Cas9 ribonucleoprotein (RNP) complexes using a Lonza 4D 96-well electroporation system with pulse code EH115. Immediately after electroporation, 80 μl of pre-warmed media was added to each well, and cells were placed to rest for 15 min at 37° C. Treg cells were then stimulated with anti-CD3/CD28 beads at a ratio 1:1 in presence of 300UI of IL-2 per ml as detailed in Example 3. Two days after sort and electroporation, Treg cells were transduced with different CAR constructs as described in Example 3. 7 days later, cells were sorted based on the expression of EGFRt and CD3 into TCR^{KO} CAR+ or TCR+ CAR+ populations. Cells were then re-stimulated with anti-CD3/CD28 beads at a ratio 1:1 in presence of 300UI of IL-2 per ml for 5 to 6 days.

Suppression assay with TCR^{KO} CAR⁺ Tregs. Treg suppression was assessed by measuring proliferation based on [³H] thymidine incorporation. After 12 days of expansion, anti-CD3/CD28 beads were removed from the TCR^{KO} CV.28z-CAR⁺ Treg and TCR^{KO} 19.28 z-CAR⁺ Treg cultures. Cells were rested for 2 days prior suppression assay. The day before the assay, CD4⁺ T effector cells (responder cells) were thawed and kept at 37° C. 5% CO₂ overnight in presence of IL-2 30UI/ml. Round bottom 96-well plates were coated with anti-CD3 antibody at 5 mg/ml overnight and washed with 1×PBS. The day of the assay, TCR^{KO} CAR⁺ Treg populations and responder cells were washed twice to remove residual IL-2 from the media. Cells were then plated in anti-CD3 Ab coated wells at 50,000 responder cells per well and TCR^{KO} CAR⁺ Tregs were added at different Responder:Treg ratios (from 2:1 to 64:1) in presence of rather Vimentin-SA-bead, CV-SA-bead, CD19-beads or no beads. 3 days later, 20 μl of [³H] thymidine (1 μCi) were added to each well. 16 hours later, plates were frozen at −20 degrees. Plates were harvested on a Packard FilterMate Harvester and count per minute (CPM) for each well was read on a Packard TopCount Scintillation and Luminescence Counter (Perkin Elmer, Waltham, Mass.). For both assays, percent suppression was calculated as followed:

% of suppression=1−[meanCPM(Treg+Responder)/meanCPM(responder alone)]×100%.

Generation of CV-CAR-Transduced Human Tregs and Tconv

Samples, cell sorting and in vitro stimulation: Fresh whole blood units were obtained from healthy blood donors recruited from the general population at the University of California, San Francisco or provided by StemCell Technologies. PBMCs were isolated by density gradient sedimentation using Ficoll Paque medium (GE healthcare). CD4⁺ T cells were enriched by positive selection from PBMCs by magnetic cell sorting (Miltenyi Biotec). CD4⁺ T cells were then stained with fluorochrome-labeled mAb specific for CD4, CD25 and CD127 and separated by flow cytometry (FACSAria; BD Biosciences) into two subsets: CD4⁺CD25⁺CD127⁻ (CD4⁺ regulatory T cells; Tregs) and CD4⁺CD25⁻CD127⁺ (CD4⁺ T conventional cells; Tconv) at a purity higher than 97%. Sorted cell populations were then stimulated with anti-CD3/anti-CD28-coated Dynabeads (ThermoFisher Scientific) at ratio 1:1 for Tconv and 1 cells for 2 beads for Tregs in presence of interleukin-2 (IL-2; Proleukin, Prometheus Laboratories; 100 U/ml for Tconv and 300 U/ml for Tregs) in T cell media: RPMI 1640 media supplemented with 5mMHEPES, 2 mM L-glutamine, 50 mg/ml each penicillin/streptomycin (Invitrogen, Carlsbad, Calif.), 5 mM nonessential amino acids, 5 mM sodium pyruvate (Mediatech), and 10% FBS (Invitrogen). Fresh media containing IL-2 was added every 2 days and cells were split when needed.

Lentiviral transduction of the Tconv and Treg populations and transduction efficiency assessment: At day 2 after stimulation of the sorted CD4⁺ populations, cells were counted and seeded into 250,000 to 500,000 cells per well into a 24-well plate and placed at 37° C. for at least 1 hour in the incubator. A mix of viral particles and protamine sulfate (100 μg/ml) was then added to the wells to reach a multiplicity of infection (MOI) of 1 particle per cell. The cells were then spinoculated for 30 minutes at 1200×g at 32° C. The plate was then placed back at 37° C. for 90 minutes. The cells were spun down for 5 min and the inoculum media was replaced by a fresh one containing IL-2. Three days later, the efficiency of the transduction was determined by flow cytometry using the anti-EGFRt antibody. To confirm that the percentage of EGFRt⁺ cells was representative of the expression of the CAR at the cell surface of these cells, the cells were co-stained with EGFRt antibody-conjugated with APC or PE and a CV-Streptavidin(SA)-AF488 tetramer, incubated for 2 hours at 4° C. under agitation and analyzed by flow cytometry at day 4 after transduction. The CV-SA-AF488 complex was made just before the cell staining by co-incubating biotinylated CV peptide (Innovagen) with SA-AF488 conjugated (Life Technologies) at a ratio 1 SA protein for 4 biotinylated peptides for 15 min at 4° C. under agitation and by spinning down the tube at high 14000×g for 5 min.

Detection of the presence of citrullinated vimentin in synovial fluid by direct ELISA: 96-well ELISA plates were coated 0/N at 4° C. with chicken anti-vimentin Ab at 10 μg/ml. Wells were washed three times and blocked with 200 μl of 1×PBS 1% BSA for 1 h at RT and washed again. Different samples of synovial fluid supernatants diluted in PBS 1% BSA 0.1% Tween 20 were added and incubated for 1.5 h at RT. In some other wells, vimentin protein and citrullinated vimentin proteins were added instead of synovial fluid to be used as negative and positive controls respectively. Wells were washed three times. 100 μl of mouse chimeric BVCA1 IgG (with mouse CH2 and CH3 domains) at 10 μg/ml or isotype control were added. After 1 h of incubation at RT, wells were washed 3 times and a Goat anti-mouse IgG2a secondary Ab-AP conjugated (Abcam, ab98695) was added to the well at 1/1000 dilution. Wells were washed three times and 100 μl of Step pNPP substrate solution was added. Reaction was stopped with 50 μl of NaOH 3M after 45 min and absorbance was read in a microplate reader at 405 nm.

Co-culture of the CV-CAR Tregs with synovial fluid from RA patient: Synovial fluid (SF) samples from RA patients or negative control Gout patient provided by Jonathan Graf were used to assess the ability of the CV-CAR Tregs of being activated through the CAR by using a more disease-related source of citrullinated vimentin. When synovial fluid samples were in sufficient quantity, part of the fluid was diluted with 1×PBS and processed by density gradient centrifugation. The upper layer (cell-free) was collected after centrifugation and stored at −80 degrees. At day 9 after the first round of stimulation, whole synovial fluid samples and cell-free synovial fluid samples were thawed and mixed with T cell media supplemented with IL-2 at different ratios. CAR T cells were seeded in a 96-well plate at 50,000 cells per well and 200 μl of the mix of SF and T cell culture media was added in each well. After three days, cells were stained with antibodies specific for CD4, CD71, CD25 and LIVE/DEAD fixable blue stain as previously described. The percentage of cells expressing CD71 and the MFI of CD25 were obtained by flow cytometry by gating on the LIVE CD4⁺ population.

TABLE 1
Non-exhaustive list of modified proteins that represent target antigens for CAR development.

| Antigen | Modification | Disorder | Tissue | Reference(s) |
|---|---|---|---|---|
| Vimentin | Citrullination | RA^{#} | Synovium | PMID: 23536012 |
| | | AD | CNS | PMID: 15704193 |
| | | | | PMID: 26190193 |
| | | Sporadic CJD | Frontal | PMID: 20013286 |
| | | | cortex | |
| | | JIA^{#} | Synovium | PMID: 23987731 |
| | | IPF | Lung | PMID: 29287593 |
| | | RA-ILD | Lung | PMID: 29287593 |
| | | COPD | Lung | PMID: 25600626 |
| | | Liver Fibrosis | Liver | PMID: 24367216 |
| MBP | Citrullination | MS | CNS | PMID: 12832457 |
| GAD65 | Citrullination | T1D | Pancreas | PMID: 24705406 |
| | | | | PMID: 30307543 |
| Gliadin | Deamidation | Celiac Disease | Small | PMID: 28913337 |
| | | | intestine | |
| Amyloid-beta (Aβ) | Aggregation | AD | CNS | PMID: 25031638 |
| peptide | | | | PMID: 16103127 |
| | | | | PMID: 12566568 |
| | | | | PMID: 25281743 |
| | Nitration (Tyr¹⁰-Aβ) | AD | CNS | PMID: 21903077 |
| | Cyclization | AD | CNS | PMID: 18836460 |
| | (Pyroglutamate-Modified) | | | PMID: 24403873 |
| Tau | Hyperphosphorylation | AD | CNS | PMID: 29670132 |
| | Acetylation | AD | CNS | PMID: 21427723 |
| TAR DNA-binding | Phosphorylation | ALS | CNS/ | PMID: 18546284 |
| protein 43 (TDP-43) | | | Spinal Cord | PMID: 29460270 |
| | | FTLD-U | CNS | PMID: 18546284 |
| | | | | PMID: 29460270 |
| | Acetylation | ALS | Spinal Cord | PMID: 25556531 |
| Alpha-synuclein | Phosphorylation | Synucleinopathies | CNS | PMID: 11813001 |
| | | (PD, MSA, DLB) | | PMID: 21865317 |
| | Nitration | Synucleinopathies | CNS | PMID: 11062131 |
| | | (PD, MSA, DLB) | | PMID: 21865317 |

RA, Rheumatoid Arthritis; AD, Alzheimer's Disease; CJD, Creutzfeldt-Jakob disease; JIA, Juvenile idiopathic arthritis; CNS, Central Nervous System; IPF, Idiopathic Pulmonary Fibrosis; RA-ILD, Rheumatoid Arthritis associated Interstitial Lung Disease; COPD, Chronic Obstructive Pulmonary Disease; ALS, Amyotrophic Lateral Sclerosis; FTLD-U, Frontotemporal Lobar Degeneration with Ubiquitin-positive inclusions; PD, Parkinson's Disease; MSA, Multiple System Atrophy; DLB, Dementia with Lewi Bodies.
^{#}Confirmed by our in vitro data.

### REFERENCES

1. Bard, F. et al. Epitope and isotype specificities of antibodies to -amyloid peptide for protection against Alzheimer's disease-like neuropathology. Proceedings of the National Academy of Sciences 100, 2023-2028 (2003).
2. Hatami, A. et al. Monoclonal antibodies against Aβ42 fibrils distinguish multiple aggregation state polymorphisms in vitro and in Alzheimer disease brain. Journal of Biological Chemistry 289, 32131-32143 (2014).
3. Yen, S. H. et al. Alzheimer's neurofibrillary tangles contain unique epitopes and epitopes in common with the heat-stable microtubule associated proteins tau and MAP2. The American journal of pathology vol. 126, 1 (1987): 81-91.
4. Zhang, H. et al. Structural Basis for Recognition of a Unique Epitope by a Human Anti-tau Antibody. Structure 26, 1626-1634.e4 (2018).
5. Lugli, Elena B et al. Expression of citrulline and homocitrulline residues in the lungs of non-smokers and smokers: implications for autoimmunity in rheumatoid arthritis. Arthritis research & therapy vol. 17, 1 9. 20 (2015).
6. Luo, Guo et al. Autoimmunity to hypocretin and molecular mimicry to flu in type 1 narcolepsy. Proceedings of the National Academy of Sciences of the United States of America vol. 115, 52 (2018): E12323-E12332.
7. Leeming, D. et al. Novel serological neo-epitope markers of extracellular matrix proteins for the detection of portal hypertension. Alimentary Pharmacology & Therapeutics 38, 1086-1096 (2013).
8. WO2001079253A1: Extracellular matrix polynucleotides, polypeptides, and antibodies.

What is claimed is:

1. A chimeric antigen receptor (CAR) comprising an antigen specific binding domain, a hinge domain, a transmembrane domain, co-stimulatory domain, and a primary signaling domain, optionally derived from a CD3 chain domain, wherein the antigen specific binding domain specifically binds to a modified protein or peptide, a protein, a peptide or fragments thereof.

2. The CAR of claim 1, wherein the modified protein or protein comprises one or more proteins, peptides or fragments thereof derived from extracellular spaces at a site of an inflammation in a subject.

3. The CAR of claim 2, wherein the modified protein or protein in the extracellular spaces comprise collagens, laminins, fibronectins, tenascins, elastin, vitronectin, periostin, maltose-binding protein (MBP), glutamic acid decarboxylase 65-kilodalton protein (GAD65), gliadin, amyloid-beta (Aβ) peptide, Tau, TAR (transactive response) DNA-binding protein 43 (TDP-43), alpha-synuclein or combinations thereof.

4. The CAR of claim 2, wherein the modified protein, peptide or fragments thereof are modified by one or more processes comprising: acetylation, acylation, ADP-ribosylation, amidation, cross-linking, cyclization, disulfide bond formation, demethylation, formation of cysteine, formation of pyroglutamate, formylation, gamma-carboxylation, glycosylation, GPI anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins or combinations thereof.

5. The CAR of claim 2, wherein the modified protein, peptide or fragments thereof are modified by attachment of one or more molecules comprising: a flavin, a heme moiety, a nucleotide, a nucleotide derivative, a lipid, a lipid derivative, a phosphotidylinositol, or combinations thereof.

6. The CAR of claim 1, wherein the antigen specific binding domain comprises an antibody, antibody fragment or aptamer.

7. The CAR of claim 6, wherein the antibody fragment is a single chain fragment.

8. The chimeric antigen receptor of claim 7, wherein the single chain fragment is a single chain variable fragment (scFv).

9. The chimeric antigen receptor of claim 1, wherein the co-stimulatory domain comprises a CD28 or a 41BB polypeptide.

10. An isolated T cell that is modified to express: a chimeric antigen receptor (CAR) comprising an antigen binding domain linked to at least one co-stimulatory domain and a primary signaling domain, optionally derived from a CD3 chain domain, wherein the antigen binding domain specifically binds to an modified protein present in the extracellular space of the inflammatory lesion.

11. The isolated T cell of claim 10, wherein the modified protein comprises one or more proteins, peptides or fragments thereof derived from extracellular spaces at a site of an inflammation in a subject.

12. The isolated T cell of claim 11, wherein the modified protein, peptide or fragments thereof are modified by one or more processes comprising: acetylation, acylation, ADP-ribosylation, amidation, cross-linking, cyclization, disulfide bond formation, demethylation, formation of cysteine, formation of pyroglutamate, formylation, gamma-carboxylation, glycosylation, GPI anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins or combinations thereof.

13. The isolated T cell of claim 11, wherein the modified protein, peptide or fragments thereof are modified by attachment of one or more molecules comprising: a flavin, a heme moiety, a nucleotide, a nucleotide derivative, a lipid, a lipid derivative, a phosphotidylinositol, or combinations thereof.

14. The isolated T cell of claim 11, wherein the modified protein or protein in the extracellular spaces comprise collagens, laminins, fibronectins, tenascins, elastin, vitronectin, periostin, maltose-binding protein (MBP), glutamic acid decarboxylase 65-kilodalton protein (GAD65), gliadin, amyloid-beta (Aβ) peptide, Tau, TAR (transactive response) DNA-binding protein 43 (TDP-43), alpha-synuclein or combinations thereof.

15. The isolated T cell of claim 10, wherein the CAR antigen specific binding domain comprises an antibody, antibody fragment or aptamer.

16. The isolated T cell of claim 15, wherein the antibody fragment is a single chain fragment.

17. The isolated T cell of claim 16, wherein the single chain fragment is a single chain variable fragment (scFv).

18. The isolated T cell of claim 10, wherein the co-stimulatory domain comprises a CD28 or a 41BB polypeptide.

19. A method of treating a subject diagnosed with an inflammatory disease, comprising:
i. identifying a modified protein present in the extracellular space of the inflammatory lesion;
ii. generating a chimeric antigen receptor (CAR) that binds to the modified protein of step (i);
iii. expressing the CAR of step (ii) in an isolated T cell;
iv. administering the isolated T of step (iii) to a subject.

20. The method of claim 19, comprising an antigen specific binding domain, a hinge domain, a transmembrane domain, co-stimulatory domain, and a primary signaling domain, optionally derived from a CD3 chain domain, wherein the antigen specific binding domain specifically binds to the modified protein present in the extracellular space of the inflammatory lesion.

21. The method of claim 19, wherein the modified protein comprises one or more proteins, peptides or fragments thereof derived from extracellular spaces at a site of an inflammation in a subject.

22. The method of claim 21, wherein the modified protein, peptide or fragments thereof are modified by one or more processes comprising: acetylation, acylation, ADP-ribosylation, amidation, cross-linking, cyclization, disulfide bond formation, demethylation, formation of cysteine, formation of pyroglutamate, formylation, gamma-carboxylation, glycosylation, GPI anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins or combinations thereof.

23. The method of claim 21, wherein the modified protein, peptide or fragments thereof are modified by attachment of one or more molecules comprising: a flavin, a heme moiety, a nucleotide, a nucleotide derivative, a lipid, a lipid derivative, a phosphotidylinositol, or combinations thereof.

24. The method of claim 21, wherein the modified protein or protein in the extracellular spaces comprise collagens, laminins, fibronectins, tenascins, elastin, vitronectin, periostin, maltose-binding protein (MBP), glutamic acid decarboxylase 65-kilodalton protein (GAD65), gliadin, amyloid-beta (Aβ) peptide, Tau, TAR (transactive response) DNA-binding protein 43 (TDP-43), alpha-synuclein or combinations thereof.

25. The method of claim 19, wherein the antigen specific binding domain comprises an antibody, antibody fragment or aptamer.

26. The method of claim 25, wherein the antibody fragment is a single chain fragment.

27. The method of claim 26, wherein the single chain fragment is a single chain variable fragment (scFv).

28. The method of claim 19, wherein the co-stimulatory domain comprises a CD28 or a 41BB polypeptide.

29. The method of claim 19, wherein the isolated T cells are autologous cells.

30. The method of claim 19, further comprising administering to the subject one or more anti-inflammatory agents and/or therapeutic agents.

31. The method of claim 30, wherein the anti-inflammatory agents comprise one or more antibodies which specifically bind to pro-inflammatory cytokines, anti-inflammatory cytokines or chemokines or receptors thereof, nonsteroidal anti-inflammatory drugs (NSAIDs), steroidal anti-inflammatory drugs, or combinations thereof.

32. The method of claim 31, wherein the antibodies are anti-TNFα, anti-IL-6 or combinations thereof.

33. The method of claim 31, wherein the anti-inflammatory cytokines or chemokines or receptors thereof, comprise interleukin (IL)-1 receptor antagonist, IL-4, IL-6, IL-10, IL-11, IL-13 or combinations thereof.

34. An expression vector encoding the CAR of claim 1.

35. An isolated cell comprising the expression vector of claim 34.

36. A pharmaceutical composition comprising the CAR of claim 1, the isolated T cell of claim 19, the expression vector of claim 34, or the isolated cell of claim 35.
